(19) United States
(12) Patent Application Publication
(10) Pub. No.: US 2025/0371705 A1
(43) Pub. Date: Dec. 4, 2025

(54) DETERMINING TUMOR RESPONSIVENESS TO RADIOTHERAPIES FROM BIOMEDICAL IMAGES

(71) Applicants: Memorial Sloan-Kettering Cancer Center, New York, NY (US); Memorial Hospital for Cancer and Allied Diseases, New York, NY (US); Sloan-Kettering Institute For Cancer Research, New York, NY (US)

(72) Inventors: Michalis Aristophanous, New York, NY (US); Dylan Hsu, New York, NY (US); Ase Ballangrud-Popovic, New York, NY (US)

(21) Appl. No.: 19/222,864

(22) Filed: May 29, 2025

Related U.S. Application Data
(60) Provisional application No. 63/653,707, filed on May 30, 2024.

(51) Int. Cl. G06T 7/00; G06T 7/11; G06T 7/33; G16H 15/00; G16H 20/40

(52) U.S. Cl. CPC G06T 7/0012; G06T 7/11; G06T 7/337; G16H 15/00; G16H 20/40; G06T 2207/30016; G06T 2207/30096

(57) ABSTRACT

Presented herein are systems and methods of determining tumor responses in brains from administering radiotherapy. A computing system can: identify a plurality of biomedical images of a brain of a subject; perform an image registration on a first biomedical image with a second biomedical image to determine a plurality of translation parameters; generate a third biomedical image using the second biomedical image in accordance with the plurality of translation parameters, detect using an image segmentation model, (i) a first segment identifying a first region within the third biomedical image and (ii) a second segment identifying a second region within the second biomedical image; and determine a metric indicating a degree of responsiveness of a tumor in the subject to the administration of the radiotherapy to the brain, based on the first segment and the second segment.

### CROSS REFERENCES TO RELATED APPLICATIONS

This application claims the benefit of and priority to U.S. Provisional Application No. 63/653,707, titled “Determining Tumor Responsiveness to Radiotherapies from Biomedical Images,” filed May 30, 2024, which is incorporated herein by reference in its entirety.

### BACKGROUND

A computing device may process image data using computing vision techniques to provide an output.

### SUMMARY

Aspects of the present disclosure are directed to systems and methods for determining tumor responses in brains from administering radiotherapy. One or more processors coupled with memory, may: identify, for a subject diagnosed with cancer, a plurality of biomedical images of a brain of the subject, the plurality of biomedical images including: (i) a first biomedical image acquired at a first time instance, the first biomedical image having (a) a first portion corresponding to a first structure of the brain, (b) a second portion corresponding to a second structure of the brain, and (c) a first region of interest (ROI) corresponding to a tumor within the brain at the first time instance; (ii) a second biomedical image of the brain acquired at a second time instance subsequent to an administration of radiotherapy to the brain, the second biomedical image having (a) a third portion corresponding to the first structure, (b) a fourth portion corresponding to the second structure, and (b) a second ROI corresponding to the tumor within the brain at the second time instance; perform an image registration on the first biomedical image with the second biomedical image to determine a plurality of translation parameters, based on (i) a first correspondence between the first portion and the third portion for the first structure and (ii) a second correspondence between the second portion and the fourth portion for the second structure; generate a third biomedical image using the second biomedical image in accordance with the plurality of translation parameters; detect, using an image segmentation model, (i) a first segment identifying the first ROI within the third biomedical image and (ii) a second segment identifying the second ROI within the first biomedical image; determine a metric indicating a degree of responsiveness of the tumor in the subject to the administration of the radiotherapy to the brain, based on the first segment and the second segment; and store, using one or more data structures, an association between the subject and the metric.

In some embodiments, the one or more processors may: identify the tumor as targeted for the administration of radiotherapy from a plurality of treatment parameters defining the administration of the radiotherapy to the tumor prior to the second time instance; and determine a second metric indicating a dose of the radiotherapy on the tumor based on at least one of the plurality of treatment parameters, the first segment and or the second segment, responsive to identifying the tumor as targeted for the administration of radiotherapy, wherein the one or more processors may store the association among the subject, the tumor, the metric indicating the degree of responsiveness, and the second metric indicating the dose of the radiotherapy.

In some embodiments, the one or more processors may: identify the tumor corresponding to at least one of the first segment or the second segment as not targeted for the administration of radiotherapy, using a plurality of treatment parameters defining the administration of the radiotherapy; determine a second metric indicating a dose of the radiotherapy on the tumor based on at least one of the first segment or the second segment, responsive to identifying the tumor as not targeted for the administration of radiotherapy; and store the association among the subject, the tumor, the metric indicating the degree of responsiveness, and the second metric indicating the dose of the radiotherapy. In some embodiments, the one or more processors may generate a report identifying the tumor and the metric indicating the degree of responsiveness of the tumor to the administration of the radiotherapy; and provide for presentation, the report identifying the tumor and the metric.

In some embodiments, the one or more processors may generate a first plurality of translation parameters based on a moment of intensities for the second biomedical image; determine a second plurality of translation parameters to align the third portion in the second biomedical image with the first portion in the first biomedical image, the first portion and the third portion each corresponding to a respective contour of a parenchyma of the brain in the subject; modify the second plurality of translation parameters to generate the plurality of translation parameters to correspond the fourth portion of the second biomedical image with the second portion of the first biomedical image, the third portion and the second portion each corresponding to a lateral ventricle of the brain in the subject. In some embodiments, the one or more processors may detect, using one or more image segmentation models, (i) the first portion and the second portion from the first biomedical image and (ii) the third portion and the fourth portion from the second biomedical image. In some embodiments, the one or more processors may determine the metric based on a difference in longitudinal size between the first segment and the second segment.

In some embodiments, the image segmentation model is established using a plurality of examples, each example of the plurality of examples including (i) a respective sample biomedical image of a corresponding brain of a respective subject having a respective ROI corresponding to a respective tumor in the corresponding brain and (ii) a respective annotation identifying a corresponding segment identifying the respective ROI. In some embodiments, the tumor of the subject as identified by at least one of the first segment or the second segment may be administrated with the radiotherapy at a third time instance subsequent to the second time instance. In some embodiments, the cancer associated with the tumor includes a metastasized cancer. For example, the metastasized cancer may include at least one of colorectal cancer, lung cancer, breast cancer, ovarian cancer, prostate cancer, uterine cancer, or thyroid cancer. In some embodiments, the radiotherapy further includes at least one of, stereotactic radiosurgery (SRS), a brachytherapy, a proton radiotherapy, or a whole brain radiation therapy (WBRT).

In some embodiments, the one or more processors may determine a second metric indicating a brain metastasis velocity (BMV) of the tumor within the brain of the subject across the first time instance and the second time instance, based on the first segment and the second segment. The one or more processors may store the association among the subject, the tumor, the metric indicating the degree of responsiveness, and the second metric indicating the BMV of the brain metastases.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1: Schematic showing an overview of the METRO software. Planning and follow-up MR scans are obtained from the PACS. Treatment plans with calculated dose and CT scans are obtained from the TPS.

FIG. 2: Linear regression of size response for treated BMs that were still visible on follow-up, as measured automatically by METRO versus manually. One outlier with diameter change greater than +200% is suppressed.

FIGS. 3A and 3B: Distributions of residual errors for the size changes measured by automatic METRO method for the BMs still visible at follow-up. The residual is the difference of the percent change measured with METRO minus that measured manually. 3A: 3D longest diameter. 3B: equivalent sphere diameter.

FIGS. 4A-4C: This 2 cm enhancing lesion in the right frontal lobe was treated with 27 Gy in three fractions using the MR scan. The planning target volume (PTV) is shown in red, and the longitudinal AI segmentations in blue. The lesion size is decreasing at all follow-up MR images with the last MR. The METRO report shows both the 3D longest diameter and the volume based on the AI segmentation at each MR.

FIGS. 5A-5D: A lesion in the right parietal lobe is targeted with single-fraction SRS using the MR for treatment planning. There is not much size change in the first nine months after treatment, but a year and a half after treatment, at the MR scan, the lesion size is increasing. The increase in size may be evaluated for potential progression or necrosis.

FIGS. 6A-6E: This patient received three courses of SRS elsewhere in the brain, followed by WBRT (Rx 30 Gy). Then, the METRO workflow identified a new lesion in the right parietal lobe at the MR, which is increasing in size at subsequent follow-up.

FIG. 7: Number of brain metastasis patients and lesions treated at our institution per year. A slight decrease occurred in 2020-2021 due to the pandemic.

FIG. 8: Visualization of some more challenging longitudinal registrations for three patients (top, middle, bottom) with significant changes to lateral ventricles. The pre-treatment MR scans are in grayscale and the follow-up scans are in red. From top to bottom, the time intervals between scans were 1299, 1128, and 1053 days, and the average registration errors (Points A through G) were 1.4, 0.7, and 2.9 mm respectively.

FIGS. 9A-9C: Patient underwent resection and radiation for a large metastasis at an outside hospital, followed by 2 courses of radiation elsewhere in the brain at the center. The tracking software identified local enhancement as a recurrence with subsequent progression. This is a post-operative cavity that was radiated at an outside hospital before patient received 2 courses of SRS/HYPO to other brain lesions at our institution. The spillage dose from these treatment to the cavity is indicated with gray dash-dotted lines. METRO identified new enhancement in the cavity and shows increasing size over time. The increase in size may be evaluated for potential progression or necrosis.

FIGS. 10A-10E: A right cerebellar lesion was targeted with single-fraction SRS, but follow-ups show increased lesion size after treatment up to the MR scan, then a decreased size at the latest follow-up MR. The temporal change in volume is possibly treatment effects. There was prior SRS elsewhere in the brain, but prior mean dose to this area was negligible.

FIG. 11 illustrates a block diagram of an example system to determine tumor responses in brains from administering radiotherapy

FIG. 12 illustrates a block diagram of an example process for identifying, for a subject diagnosed with cancer, a plurality of biomedical images of a brain of the subject.

FIG. 13 illustrates a block diagram of an example process for performing an image registration.

FIG. 14 illustrates a block diagram of an example process for detecting a segment identifying a region of interest.

FIG. 15 illustrates a block diagram of an example process for determining a responsiveness metric.

FIG. 16 illustrates an illustrative flow diagram of an example method for determining tumor responses in brains from administering radiotherapy.

FIG. 17 illustrates a simplified block diagram of an example of a representative server system, client computing system, and network usable to implement certain embodiments of the present disclosure.

### DETAILED DESCRIPTION

Following below are more detailed descriptions of various concepts related to, and embodiments of, systems and methods for determining tumor responses in brains from administering radiotherapy. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

Section A describes Automatically tracking brain metastases after stereotactic radiosurgery.

Section B describes a system and method for determining tumor responses in brains from administering radiotherapy.

Section C describes a network environment and computing environment which may be useful for practicing various computing related embodiments described herein.

A. Automatically Tracking Brain Metastases after Stereotactic Radiosurgery

Patients with brain metastases (BMs) are surviving longer and returning for multiple courses of stereotactic radiosurgery. BMs are monitored after radiation with follow-up magnetic resonance (MR) imaging every 2-3 months. The present application provides solutions to automatically track BMs on longitudinal imaging and quantify the tumor response after radiotherapy.

The techniques disclosed herein, sometimes referred to as METRO process (MEtastasis Tracking with Repeated Observations), can automatically process patient data and track BMs. For example, a longitudinal intrapatient registration method for T1 MR post-Gd can be achieved and validated on 20 patients. Detections and volumetric measurements of BMs can be obtained from a deep learning model. BM tracking can be validated on 32 separate patients by comparing results with manual measurements of BM response and radiologists' assessments of new BMs. Linear regression and residual analysis can be performed to assess accuracy in determining tumor response and size change.

In some examples, a total of 123 irradiated BMs and 38 new BMs can be tracked. 66 irradiated BMs can be visible on follow-up imaging 3-9 months after radiotherapy. Comparing their longest diameter changes measured manually vs. METRO, the Pearson correlation coefficient can be 0.88 (p<0.001); the mean residual error can be −8±17%, according to some examples. The mean registration error can be 1.5±0.2 mm, according to some examples.

Automatic, longitudinal tracking of BMs using deep learning methods can be achieved. In particular, the system METRO fulfills a need to automatically track and quantify volumetric changes of BMs prior to, and in response to, radiation therapy.

Brain metastases (BMs) are the most common form of brain tumors. It is estimated that 10-40% of all cancer patients will develop brain metastases, with an estimated 70,000-400,000 cases/year in the United States. Historically, the standard of care for patients with multiple BMs was whole brain radiation therapy (WBRT), which controls the disease for some time, but carries the possibility of cognitive side effects and reduced quality of life. In addition, delivering additional courses radiation may increase the risk of radionecrosis. Recent advances in technology have allowed treatment of multiple BMs with frameless single-fraction or hypofractionated stereotactic radiosurgery (SRS/HYPO). Evidence of reduced cognitive decline has led to the primacy of SRS/HYPO for treating BMs. Moreover, there is an increasing trend to manage multiple or recurrent BMs with multiple courses of SRS/HYPO.

Patients with BMs are typically monitored with magnetic resonance (MR) imaging performed every two-three months, including Tl post-Gd (TIC+). The complexity of longitudinally tracking cach BM and determining treatment response increases with each course of radiation or follow-up MR. Key challenges include classifying BMs as progressing, stable, or recurring, separating new BMs from treated BMs, and differentiating recurrent metastases from delayed radiation necrosis or post-treatment effects.

Limited prior work exists on automatic BM tracking. In one approach, a Jacobian operator field can be applied to detect size changes in brain metastases on longitudinal MRs, with significant challenges from new and resolved BMs and false positive detections from blood vessels. In another approach, deformable intrapatient registration can be performed to estimate BM size changes, but such an approach relied on manual segmentation of the initial tumor. BMs can be tracked with significant manual user input, but without providing a feasible or practical solution for patients with multiple BMs and multiple prior treatments. In another approach, a PACS-integrated tracking tool for tracking BMs can be used, but there are currently no available solutions which integrate automatic image registration, detection, segmentation, and tracking, and can provide information about all treated and untreated lesions in a timely manner to the radiologist and radiation oncologist.

The techniques disclosed herein can provide solutions to automatically track size and radiation dose for multiple BMs, thereby helping clinicians make the optimal care decisions. The efficacy of the techniques disclosed herein can be validated, which detects and segments all BMs using deep learning, tracks the BMs across co-registered MR timepoints, and reports the radiation dose from prior treatments.

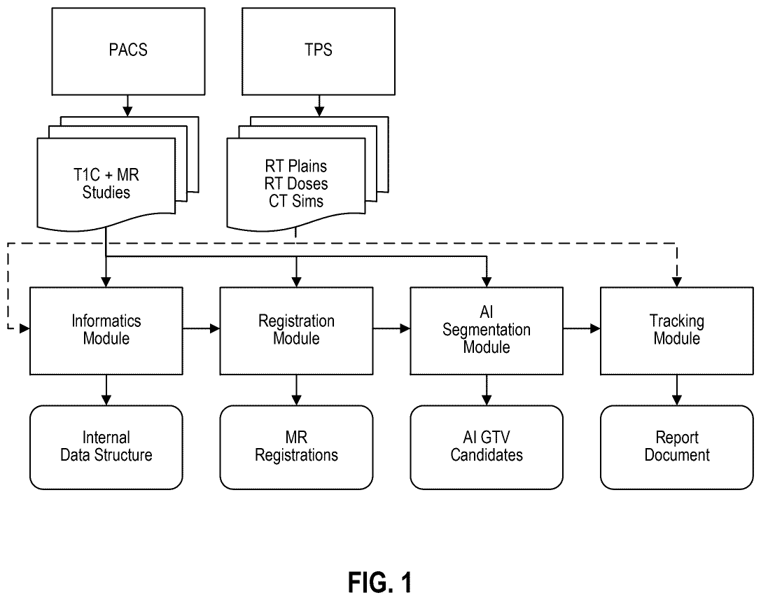

FIG. 1 illustrates an example overview of planning and follow-up MR scans obtained, in which treatment plans with calculated dose and CT scans are obtained from the TPS. In some embodiments, shown in FIG. 1 is an overview of METRO. The inputs are DICOM data (Digital Imaging and Communications in Medicine): longitudinal MR image series from the picture archiving and communication system (PACS) and DICOM-RT data from the treatment planning system (TPS). A MR series is chosen as the fixed image and rigid registration is performed with all the patient's other MRs. After registration, artificial intelligence (AI) inference is performed at each MR series using a deep convolutional neural network to produce longitudinal maps of BM gross tumor volumes (GTVs) in the fixed MR frame of reference. Next, BM tracking is performed using the treatment plan structure sets and the AI segmentations. Depending on overlap with existing GTVs from treatment plans, the detected GTVs are associated with previously treated GTVs or classified as new AI GTV candidates. The size of each GTV is tracked over time. The calculated dose distributions from prior treatment plans are overlaid on the GTVs to track the physical dose over time. Finally, a report document is generated with separate pages for each tracked BM. Raw data is also saved for downstream analysis, including tracking information such as volume and 3D longest diameter.

As months or years elapse, the patient's brain may exhibit large changes over time, including tumor control and progression, edema, midline shift, and resection cavities. Furthermore, patients who survive longer have several follow-up imaging studies. METRO co-registers all longitudinal MR imaging using a six-degree-of-freedom (6DOF) rigid registration. A multiple-stage registration procedure is used, balancing speed and accuracy, while avoiding catastrophic failures requiring manual user intervention.

The choice of fixed image for the registration depends on the treatment plans. If no co-registered treatment plans are provided, the most recent TIC+MR is the fixed image. Otherwise, it is the MR registered to the most recent treatment plan. The mutual information similarity metric was used as the cost function.

A brief description of the registration stages follows. The registration translation is initialized based on intensity moments, followed by a three-dimensional exhaustive search for initial rotation angle. Two successive stages align the entire head using gradient descent with 6DOF. Next, a fine 6DOF search is performed using only the brain parenchyma as the region of interest—this brain contour is obtained either from the structure sets of the co-registered treatment plan or an MR-based deep learning model. Lastly, a fine-tuning 6DOF search is performed using a rectangular zone centered on the lateral ventricles. After registration, all images are resampled to the fixed image with isotropic 1 mm voxel spacing.

The registration method can be validated by comparing with manually verified registrations performed by a medical physicist (e.g., on 20 BM patients). Each patient can have two TIC+ brain MR scans with a large time interval: median 392 days, interquartile range (IQR) 334-887. Seven anatomical points can be chosen, and manually located on multi-planar views of the first scan. The XYZ position of each control point can be passed through the spatial transformations from the manual and automatic registrations, and the registration error can be quantified by differences in resulting position. Further details are discussed below.

BM GTVs are automatically detected and segmented on co-registered TIC+ scans using a previously published AI model. Using GTVs authored by the radiation oncologist as ground truths, this 3D V-Net convolutional neural network can be trained (tested) on statistically independent samples of 409 (102) SRS/HYPO patients with 1345 (367) BMs. Dense evaluation of the neural network on each MR timepoint produces 3D BM probability maps in the fixed image frame of reference. To obtain discrete BM segmentations from the probability maps, a binary threshold is applied, followed by a connected-component analysis. The average patient sensitivity, false positive rate, and Dice coefficient were 95%±3%, 2.4±0.5 per patient, and 0.76 +0.03, respectively (95% confidence).

A heuristic is applied to handle instances where two BMs are conjoined into one segmentation by a sandbar or isthmus of lower probability. A morphological opening operator is applied once to each initial segmentation blob of volume V [mL]. Assuming the blob contains two spherical lesions each of volume roughly V/2, an adaptive ball kernel radius of 0.4 rₛₚₕₑᵣₑ is chosen, where

$${r}_{sphere} [ cm ] = {( \frac{3 v}{4 π} )}^{\frac{1}{3}} .$$

If the result has two separate parts, they are now each counted as separate BM detections; otherwise, the original component is kept.

After registration and AI segmentation, METRO tracks BMs over time. First, consider physician-authored GTVs with radiation prescriptions from prior treatment plans. Each expert GTV from the structure sets is rasterized, then expanded by a default 1 mm margin for longitudinal tracking. At each timepoint, any segmentation components that overlap with this expanded GTV are found; their union constitutes the tracked lesion on that scan. This search is applied to all scan time-points-BMs are tracked and measured even at MRs preceding treatment. Measurements of (non-expanded) GTV volume and 3D longest diameter (3LD) at each timepoint are computed and saved. Longitudinal size changes are measured using AI segmentations.

BMs which are newly appeared or not prescribed treatment are also tracked. To mitigate false positive detections, an increased detection probability threshold (80%) can be to identify such lesions. METRO checks each MR timepoint and finds new or untargeted AI GTVs that do not overlap with AI GTVs found at previous timepoints. Like the expert GTV tracking, these AI GTVs are tracked on past and future timepoints, using the expansion margin to check for overlap with AI segmentations. False negative, true positive, and false positive detections can be identified based on clinical radiology reports. The radiologist's axial 2D diameter measurements can be recorded for false negatives. False positives can be measured using 3LD.

Next, dose metrics are computed to each BM for patients with prior treatment data. This includes BMs not targeted for radiation by a given SRS/HYPO or WBRT plan, using the BM segmentations and the calculated dose images. Dose tracking is handled slightly differently for targeted and untargeted lesions. For linac-based SRS plans, a targeted lesion is defined as an expert GTV which has an associated planning target volume and a mean physical dose sum >10 Gy. Otherwise, it is an untargeted BM which received spillage dose. Using the original GTV volume that initiated the tracking, physical dose metrics are recorded for each treatment, including fractionation, dose delivered to 99% volume (D99%), and mean dose.

In some examples, 32 patients with BMs can be retrospectively identified under a patient consent waiver. They were treated in 2018-2021 with VMAT SRS/HYPO on linear accelerators using multiple arcs, couch rotations, and optical surface monitoring. Patients were imaged with TIC+MR prior to treatment and received regular follow-up imaging (183 total scans). METRO can be used to longitudinally track each of the 187 lesions treated within this cohort. 53 lesions were excluded due to lack of follow-ups within a chosen time window of 90-270 days. Three resection cavities, one skull lesion, and seven missing PTVs were also excluded. For the remaining 123 lesions, the follow-up MR closest to 180 days post-treatment was chosen. Median time between treatment start date and the chosen follow-up was 185 days (IQR 149.5-196).

To evaluate the accuracy measuring the size changes of tracked BMs, the pre-treatment 3LD and volume for each lesion was compared to the size at follow-up. The percent changes of 3LD and equivalent sphere diameter (ESD) between pre-treatment and follow-up scans can be calculated. To validate the tracking performance, a trained operator contoured the same 123 BMs on the pre-treatment and follow-up MR scans using MIM Maestro 6. Volume and longest diameter measurements can be extracted from each manual contour. The ESD can be derived from the manual and automatic volume measurements. 3LD and ESD can be compared between METRO and manual measurements using linear regression and the Pearson correlation coefficient. For percent size changes, the residual error can be defined as the software observation minus the human observation, with size changes determined either by 3LD or ESD. Distributions of the size change residual errors can be analyzed for both size metrics.

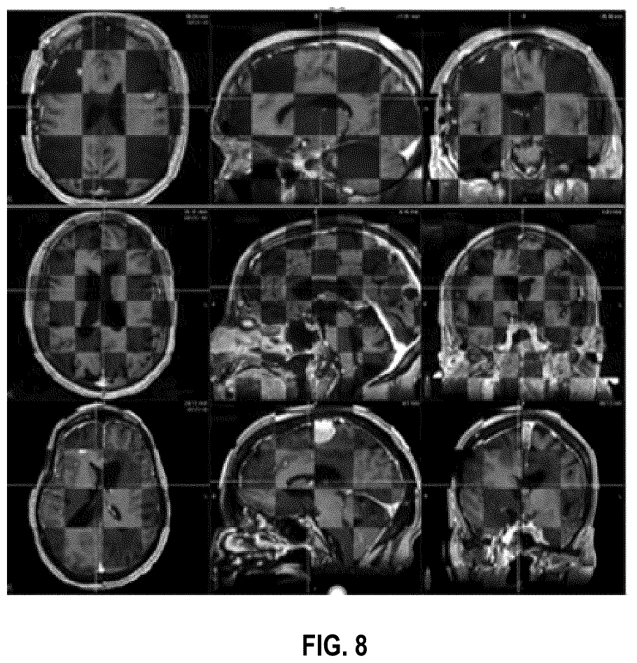

Evaluating the registrations qualitatively, differences between scan timepoints can be handled by the registration method, including ventricle size, edema, tumor progression, tumor response, and motion and susceptibility artifacts. No BM tracking failures are caused by registration uncertainty. The spatial shifts between manual and automatic registration at each anatomical control point can be assessed to quantify the registration accuracy. In some examples, the average shift per point in milli-meters was: Point A, 1.8±0.7; Point B, 1.2±0.5;Point C, 1.3±0.5; Point D, 1.6±0.6; Point E, 1.7±0.7; Point F: 1.2±0.5; Point G: 1.3±0.5(95% confidence). The average shift across all points can be 1.5±0.2 mm (95% confidence) with median 1.1 mm (IQR 0.6-1.6). Registration examples for three patients with time intervals of about three years are shown in FIG. 8.

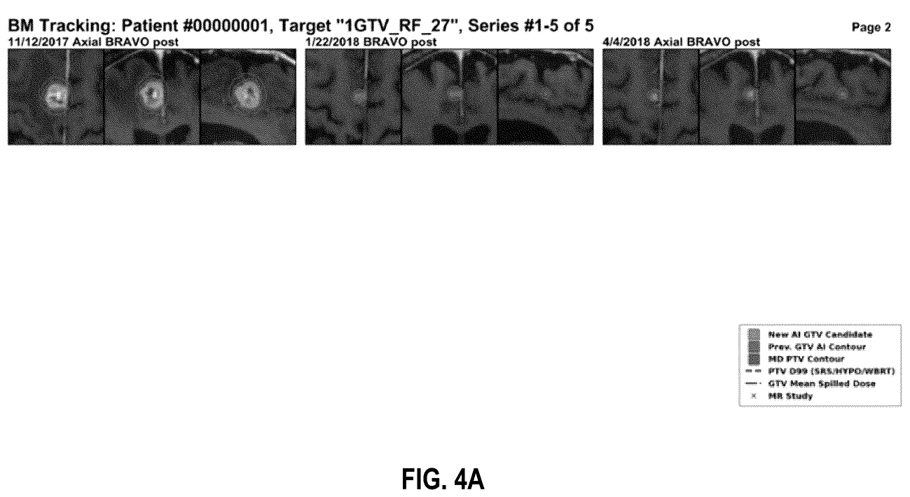

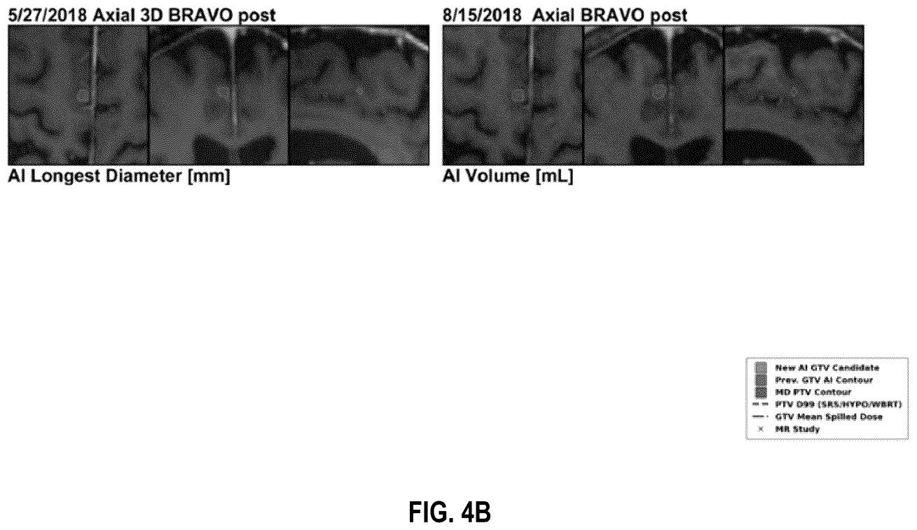

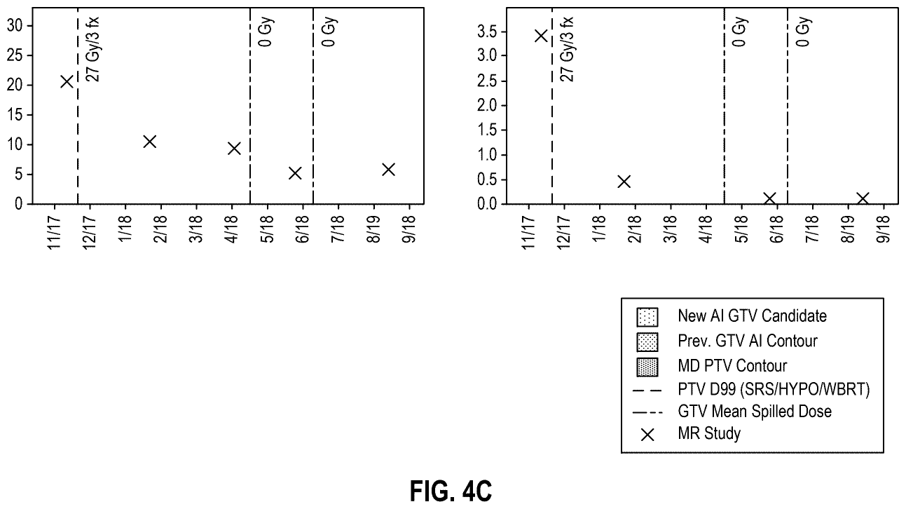

Several example reports are shown for anonymized patients. FIGS. 4A and 4B illustrate example tracking images, more specifically, a larger enhancing lesion in the right frontal lobe treated with 27 Gy over three fractions, and the report correctly identifies that the lesion responded to treatment on follow-up images. This 2 cm enhancing lesion in the right frontal lobe was treated with 27 Gy in three fractions using the MR scan from Nov. 12, 2017 for planning. The planning target volume (PTV) is shown in red, and the longitudinal AI segmentations in blue. The lesion size is decreasing at all follow-up MR images with the last MR from Aug. 15, 2018. The METRO report shows both the 3D longest diameter and the volume based on the AI segmentation at each MR.

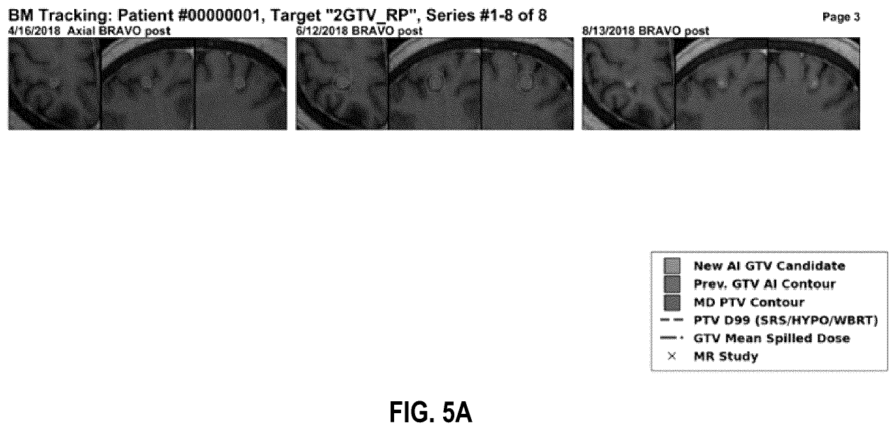

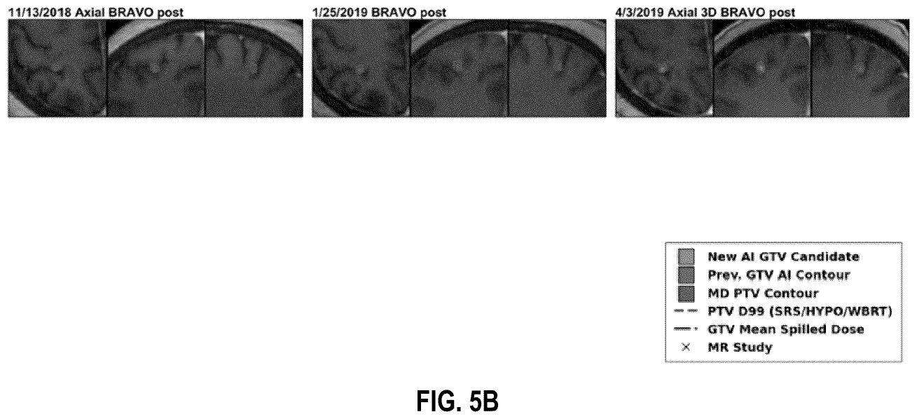

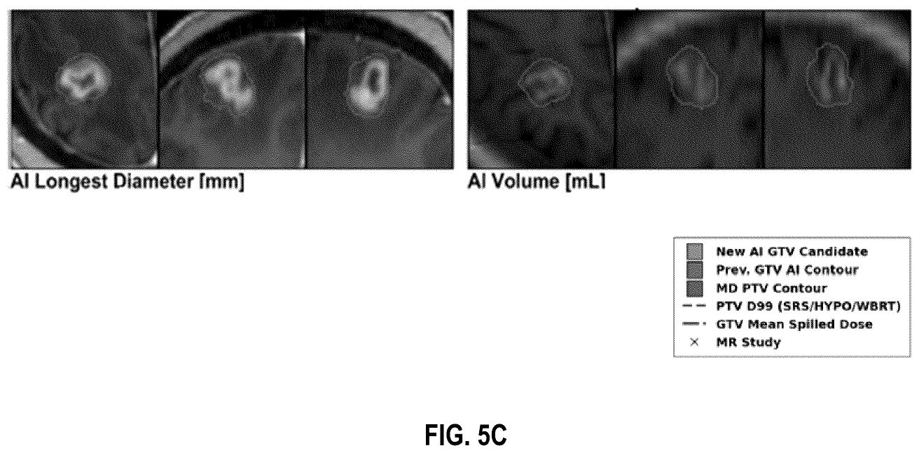

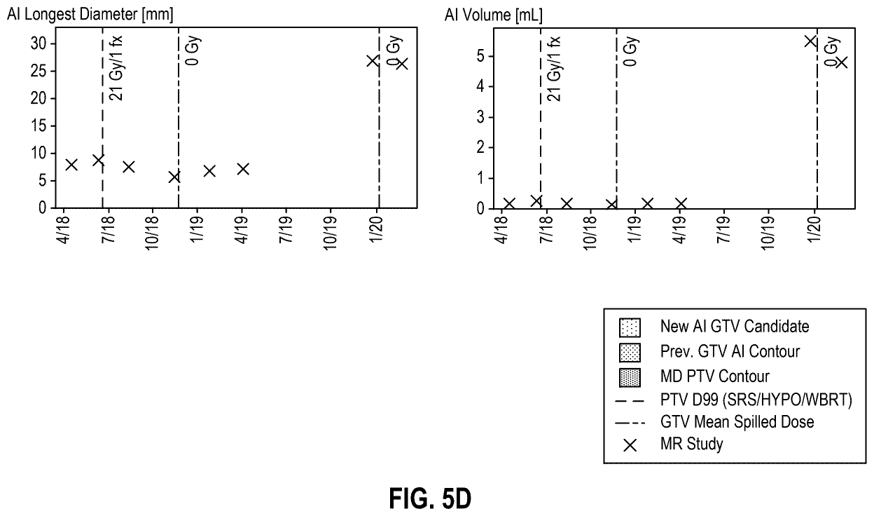

FIGS. 5A-C illustrate example tracking images, more specifically a BM in the right parietal lobe which was treated with 21 Gy (single-fraction) and is stable for some time, but exhibits an increase in size over one year later. A lesion in the right parietal lobe is targeted with single-fraction SRS using the MR from Jun. 12, 2018 for treatment planning. There is not much size change in the first nine months after treatment, but a year and a half after treatment, at the MR scan from Dec. 24, 2019, the lesion size is increasing. The increase in size may be evaluated for potential progression or necrosis.

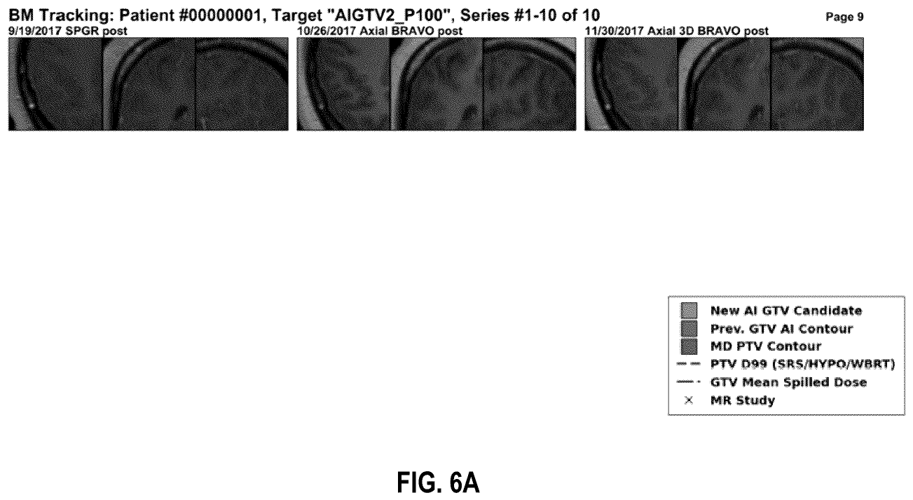

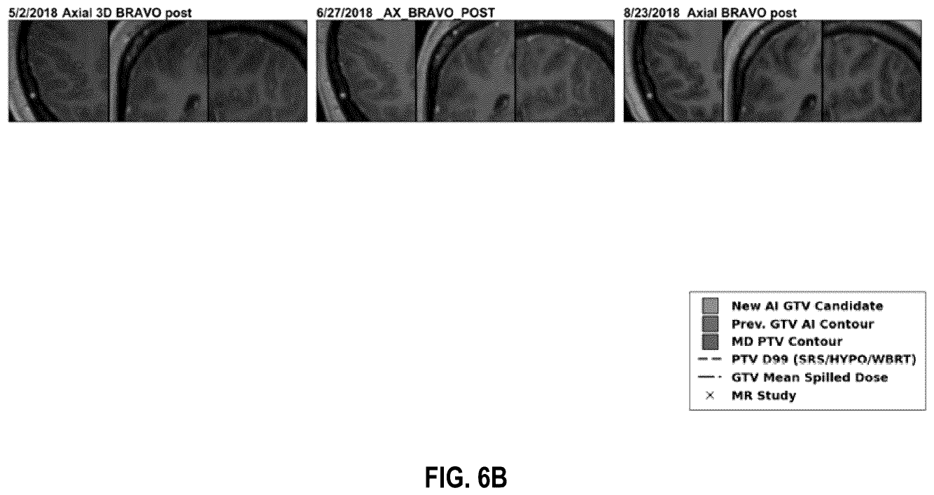

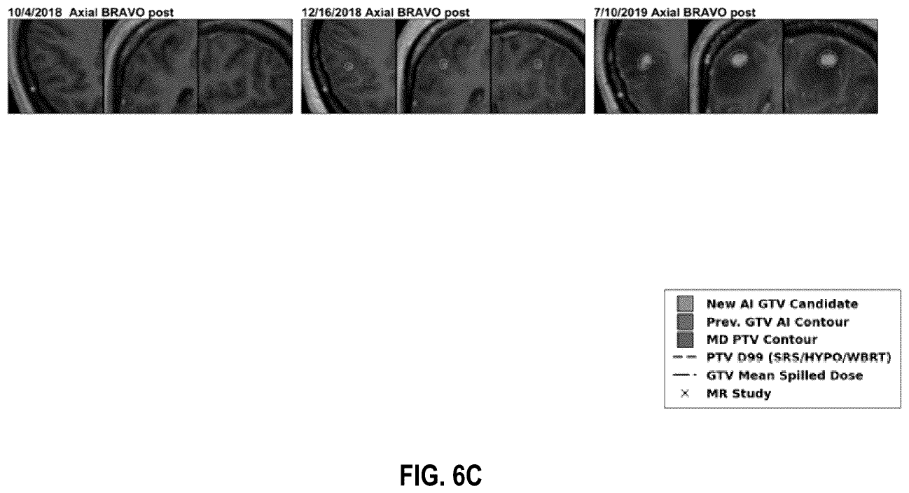

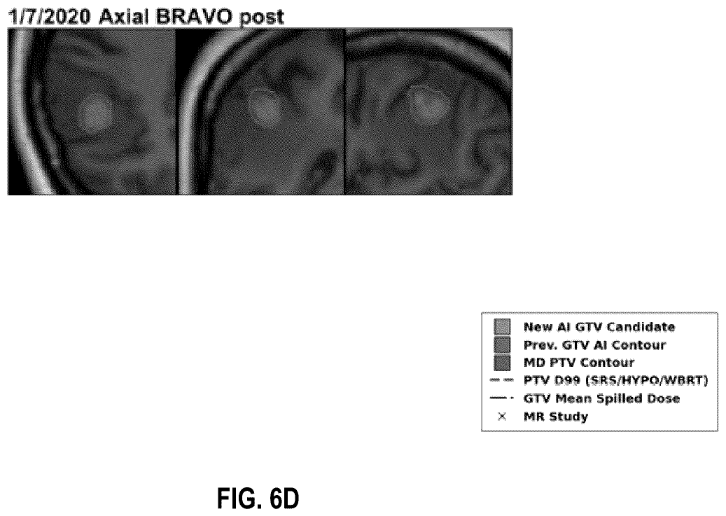

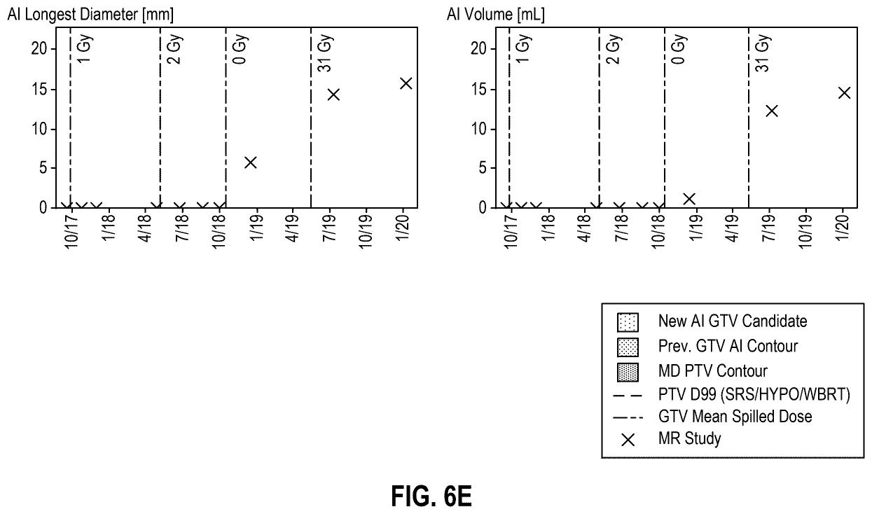

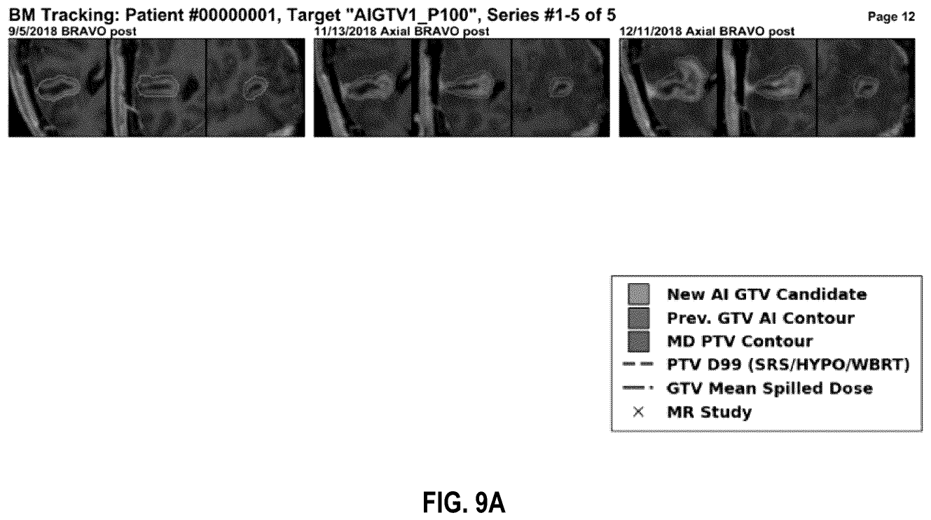

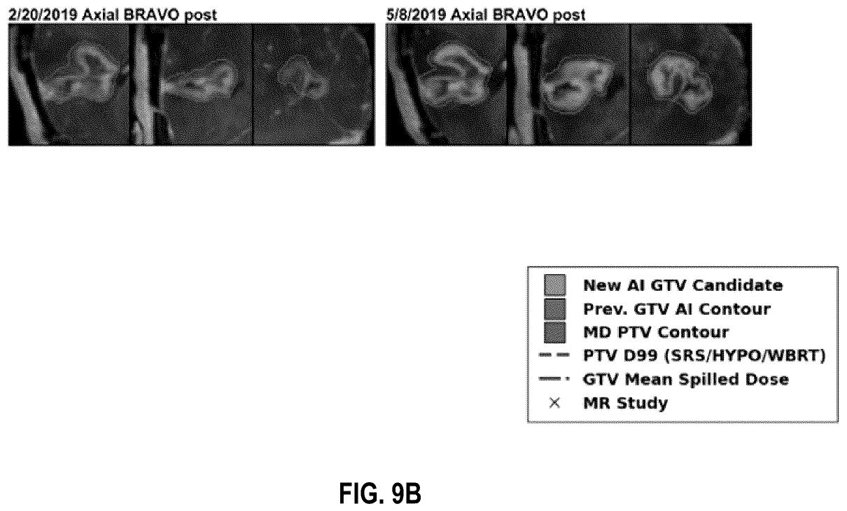

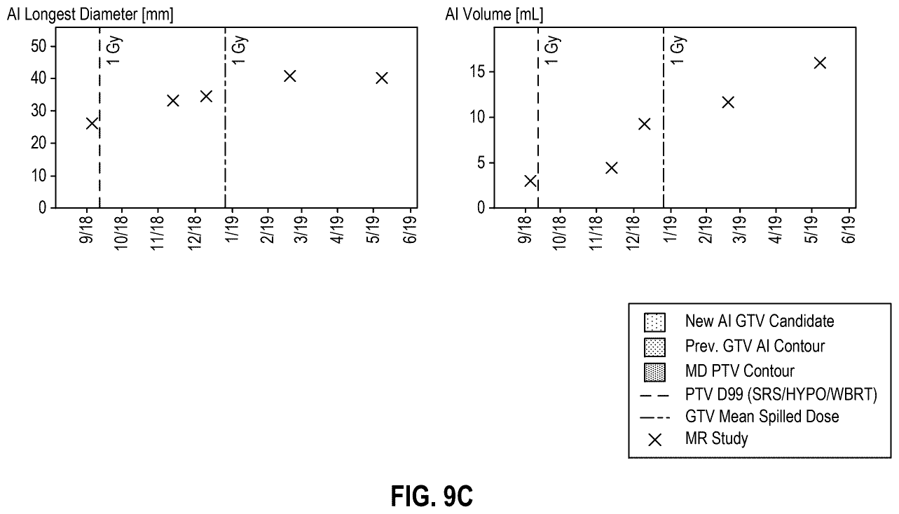

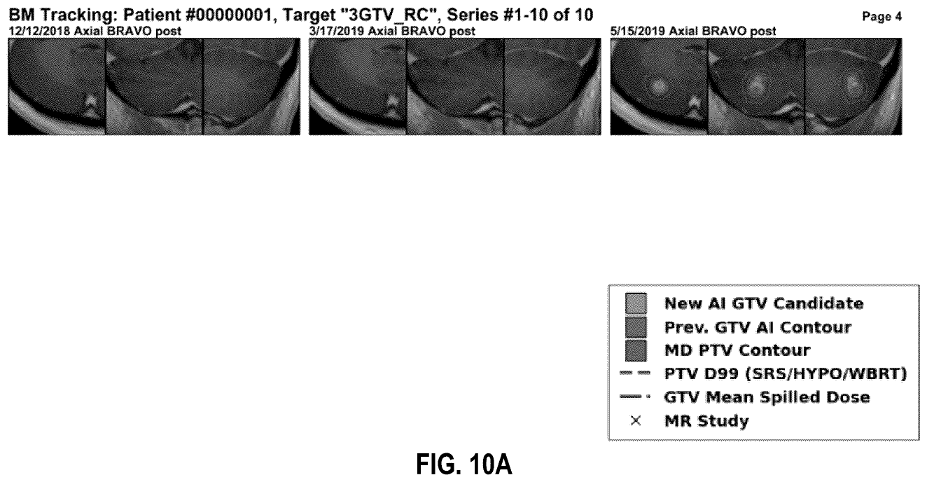

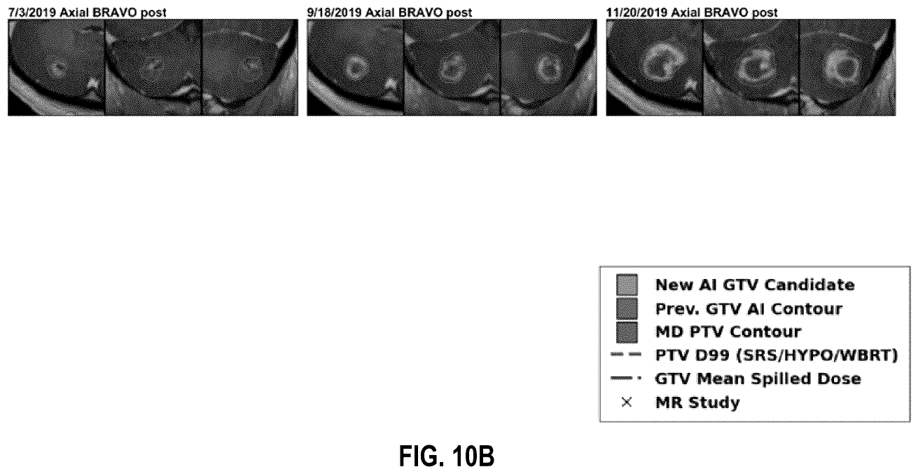

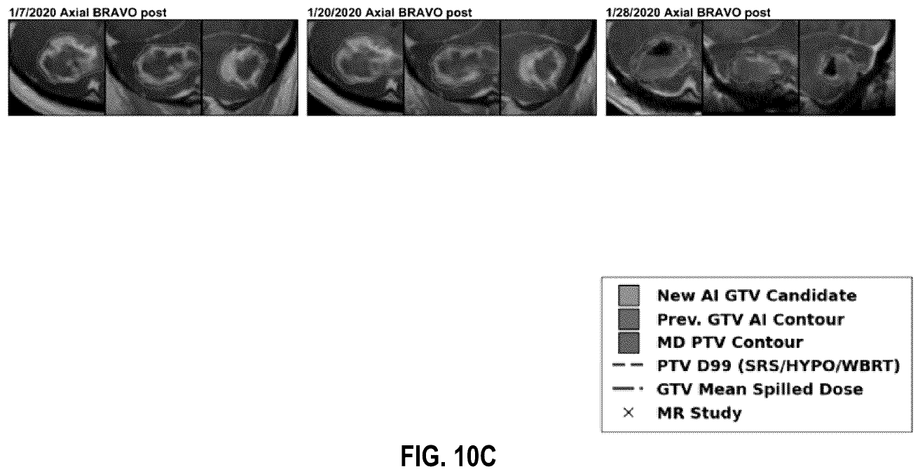

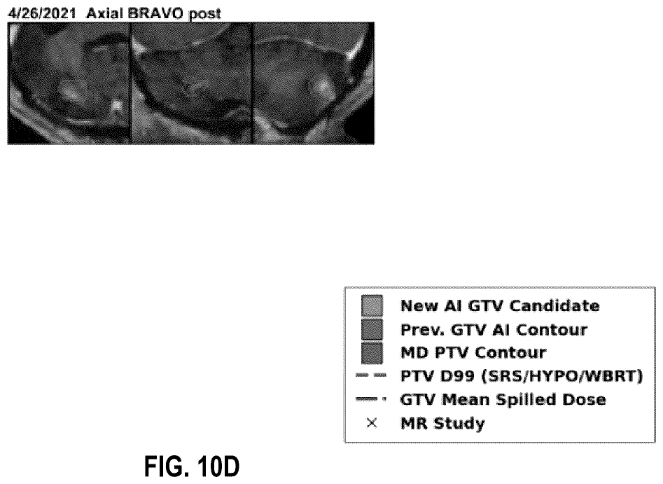

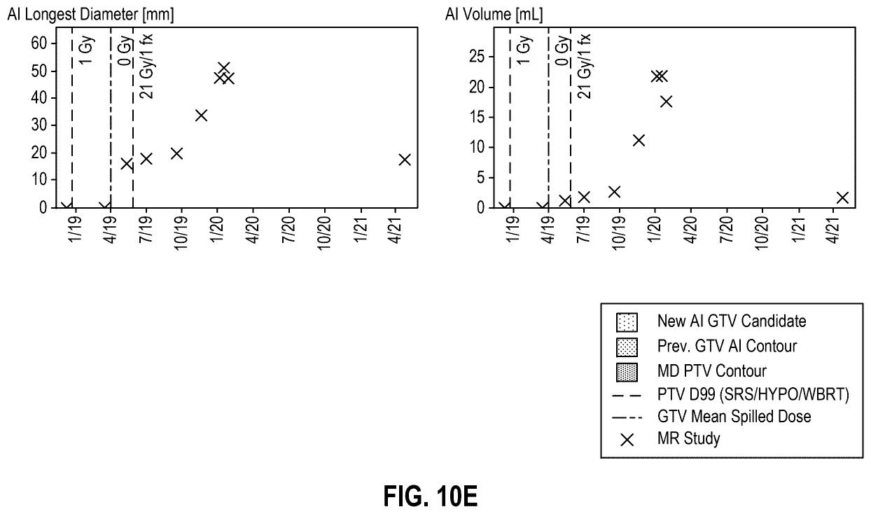

FIGS. 6A-E illustrate example tracking images, more specifically a new BM identified by METRO in the right parietal lobe after four previous courses of radiation treatments to other BMs. See FIGS. 9 and 10 for additional examples. This patient received three courses of SRS elsewhere in the brain, followed by WBRT (Rx 30 Gy). Then, the METRO workflow identified a new lesion in the right parietal lobe at the Jul. 10, 2019 MR, which is increasing in size at subsequent follow-up on Jan. 7, 2020.

72% (38/54) of new or unirradiated BMs were detected in the longitudinal tracking dataset. 92 false positive new lesions were tracked (0.5/scan). The median sizes of true positive, false negative, and false positive detections of unirradiated BMs were respectively: 0.8 cm (IQR 0.6−1.2), 0.5 cm (IQR 0.3−0.7), and 0.7 cm (IQR 0.6−0.9). The median initial size of all tracked lesions was 0.9 cm (IQR 0.6−1.3). The isthmus rule was applied 64 times (0.3/scan) and affected measurements of 15 irradiated BMs. For example, two nearby BMs in the left cerebellum and temporal lobe were measured at 7.58 and 0.73 cm³ pre-treatment, after an isthmus was removed between their conjoined segmentations.

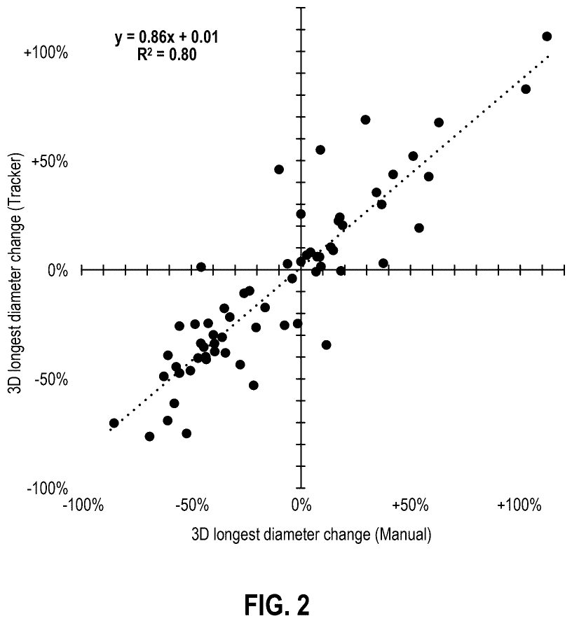

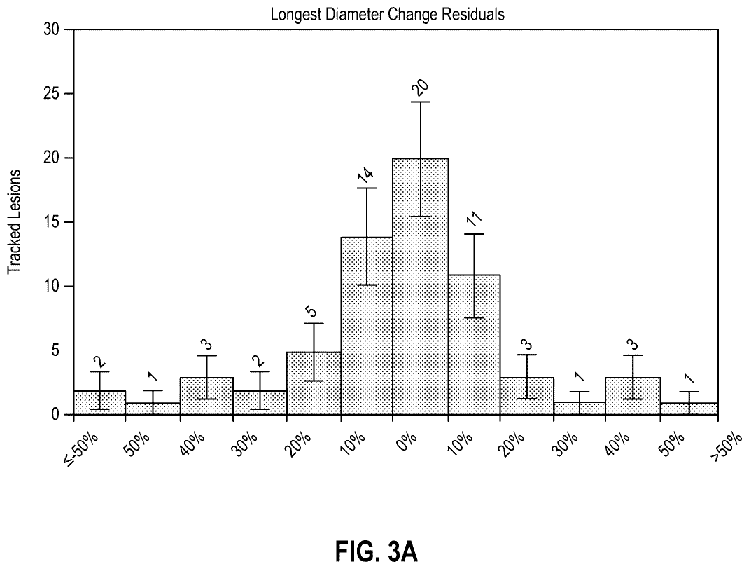

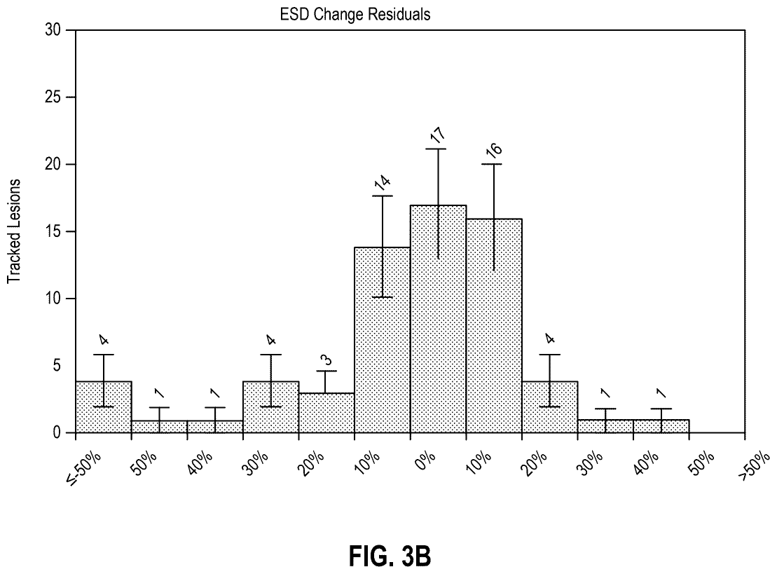

With complete BM disappearance as the quantity of interest, compared to the human observer, METRO produced 48 true positives (i.e. truly disappeared BMs), 66 true negatives, four false positives, and five false negatives. For the 66/123 BMs still visible on follow-up, linear regression (FIG. 2) shows a correlation (R²=0.80) between the size responses measured by human versus METRO. Comparing the size changes of non-disappeared lesions measured by both observers, the Pearson correlation coefficient was 0.88 for 3LD and 0.86 for ESD (p<0.001). One outlier with diameter change greater than +200% is suppressed. Distributions of the size change residual errors are shown in FIG. 3A. The mean residual of 3LD changes was −8±17% (95% confidence), with standard deviation 70%, median 2%, IQR (−7%, 12%). The mean residual of ESD changes was −12%±17% (95% confidence) with standard deviation 81%, median 2%, and IQR (−7%, 11%). The residual distributions were characterized by a central mode plus large outliers.

The METRO process was capable of reading the patient data and tracking BMs over time. The longitudinal intrapatient registration method was reliable when tested on numerous clinical cases, including those with significant time intervals and notable changes in brain anatomy, and its accuracy was sufficient for longitudinal tracking. The information gathered from each patient's imaging and treatment history is compiled in a concise, lightweight report document accessible to clinicians, and data are organized for downstream analysis.

A strong correlation was observed between the BM size changes measured by METRO and the human operator. Furthermore, positive versus negative size changes were well-differentiated by METRO—this can be observed by the lack of points in the upper-left and lower-right quadrants of FIG. 2. The distribution of residuals for size changes measured by human versus METRO (FIG. 3B) were well-centered at zero, but highly non-normal with large variance due to outliers. The residual is the difference of the percent change measured with METRO minus that measured manually. A strong correlation coefficient of 88% was observed for the measured change in longest diameter, but there is room for improvement. One potential avenue is a longitudinal segmentation model trained using multiple annotated longitudinal scans. Several cases with non-spherical BMs were noticed where the 3LD remains stable, but the BM volume is increasing. This indicates that automatic measurements of BM volume could be more clinically relevant to tumor burden than the manually—measured 2D longest diameter frequently employed in radiology practice.

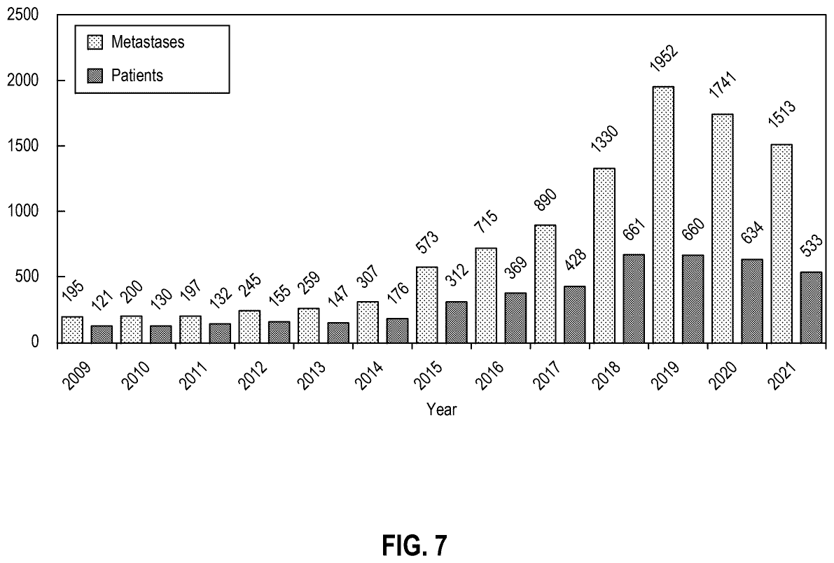

FIG. 7 illustrates an example flow chart showing number of brain metastasis patients and lesions treated per year. A slight decrease occurred in 2020-2021 due to the COVID-19 pandemic. FIG. 8 illustrates the pre-treatment MR scans (in grayscale) and the follow-up scans (in red). From top to bottom, the time intervals between scans were 1299, 1128, and 1053 days, and the average registration errors (Points A through G) were 1.4, 0.7, and 2.9 mm, respectively.

FIGS. 9A and 9B illustrate example tracking images. Here, the patient underwent resection and radiation for a large metastasis at an outside hospital, followed by 2 courses of radiation elsewhere in the brain. The tracking system identified local enhancement as a recurrence with subsequent progression. This is a post-operative cavity that was radiated at an outside hospital before patient received 2 courses of SRS/HYPO to other brain lesions at the institution. The spillage dose from these treatment to the cavity is indicated with gray dash-dotted lines. METRO identified new enhancement in the cavity and shows increasing size over time. The increase in size may be evaluated for potential progression or necrosis.

FIGS. 10A-D illustrate example tracking images, more specifically a right cerebellar lesion targeted with single-fraction SRS, but follow-ups show increased lesion size after treatment up to the MR scan on Jan. 28, 2020, then a decreased size at the latest follow-up MR on Apr. 26, 2021. The temporal change in volume is possibly treatment effects. There was prior SRS elsewhere in the brain, but prior mean dose to this area was negligible.

Varying imaging protocols and quality standards can hamper the generalizability of AI-based work such as this across different centers. Consensus guidelines are emerging for BM studies. MR imaging parameters such as pixel spacing and slice thickness can be automatically detected, but other quality aspects require standards developed by experts.

It is currently difficult to differentiate post-treatment tumor volumes from radionecrosis treatment effects. Despite the current ambiguity, post-treatment tracking of abnormal volumes associated with tumors could be an important tool to help distinguish recurrences from radio-necrotic volumes.

Manually identifying multiple, potentially irradiated BMs on longitudinal imaging is time-consuming and prone to human error. Measuring or segmenting primary and metastatic brain tumors is similarly challenging. With the aim of aiding clinical practice, this tracking workflow can be configured to integrate with the existing clinical systems. To address potential inaccuracies, a radiologist or trained operator could view and modify the results, while referencing other synchronized image series from the MR studies. The user would delete false positives and adjust inaccurate longitudinal segmentations, then an approved report would be generated. The approved structure sets would be available if further SRS treatment is indicated, and confirmed new BM appearances would be monitored automatically. These data would be invaluable not only for longitudinal retrospective studies, but also as a continuous source of BM annotations for developing better longitudinal AI models. While manual corrections do cost time, there is great potential for time savings and clinical insights as the tracking accuracy is further improved.

The automatic longitudinal tracking of brain metastases is shown to be feasible using deep learning methods. In particular, the system METRO fulfills a need to automatically track and quantify volumetric changes of brain metastases prior to, and in response to, radiation therapy. The accuracy achieved in detecting and tracking tumor volumes, excepting tumors smaller than 1 cm, appears to be adequate to support clinical workflows.

In some embodiments, exhaustive search and gradient descent strategies are both employed for the multi-stage registration. The implementation of gradient descent in Insight Toolkit v5 was used; it determines convergence by fitting the metric in a window of iterations and comparing its slope to a convergence value. Two sets of convergence conditions are defined for gradient descent: coarse and fine. The coarse criteria are: window size 50, maximum 500 iterations, convergence value 10⁻⁴. The fine criteria are: window size 50, maximum 2000 iterations, convergence value 10⁻⁶. The stage is always terminated when the maximum number of iterations is reached.

In some embodiments, the registration stages are as follows:

1. Moments-based centered transform initializer: choose initial translation parameters using a moments-based analysis of the image intensities

2. Exhaustive search for angles: freeze translation, downsampling by 3, metric sampling 10%

3. Coarse registration of whole head: downsampling by 2, metric sampling 25%, gradient descent with coarse convergence conditions

4. Fine registration of whole head: metricsampling 25%, gradient descent with fine convergence conditions

5. Fine registration of brain: Brain mask ROI, metric sampling 100%, gradient descent with fine convergence conditions

6. Fine registration of ventricle box: Ventricle box ROI, metric sampling 100%, gradient descent with fine convergence conditions

A brain mask (segmentation) is used as a region of interest for one stage of the registration metric, but it is also used to derive an approximate heuristic bounding box around the lateral ventricles. This ventricle bounding box serves as the ROI for the last stage of the registration process, aiming to emulate a box-based alignment which is practically useful in manually-guided registration. It is defined as an 80×100×50 mm³ (LR, AP, SI) box centered at 10 mm superior to the centroid of the brain mask. The brain mask is obtained from the treatment plan structure sets if available, otherwise it is produced from scratch using an AI segmentation method.

In some embodiments, the anatomical control points A-G used for validating the registration method are as follows:
Point A: midline, anterior edge of longitudinal fissure, superior edge of lateral ventricles
Point B: midline, anterior edge of brainstem, superior edge of lateral ventricles
Point C: triangulation point where the primary cerebellar fissure and the longitudinal fissure meet, inferior to lateral ventricles
Point D: bottom of left temporal lobe, posterior edge of brainstem
Point E: bottom of right temporal lobe, posterior edge of brainstem
Point F: superior/posterior edge of right lateral ventricle, superior to both lateral ventricles
Point G: superior/posterior edge of left lateral ventricle, superior to both lateral ventricles
   B. Systems and Methods for Determining Tumor Responses in Brains from Administering Radiotherapy

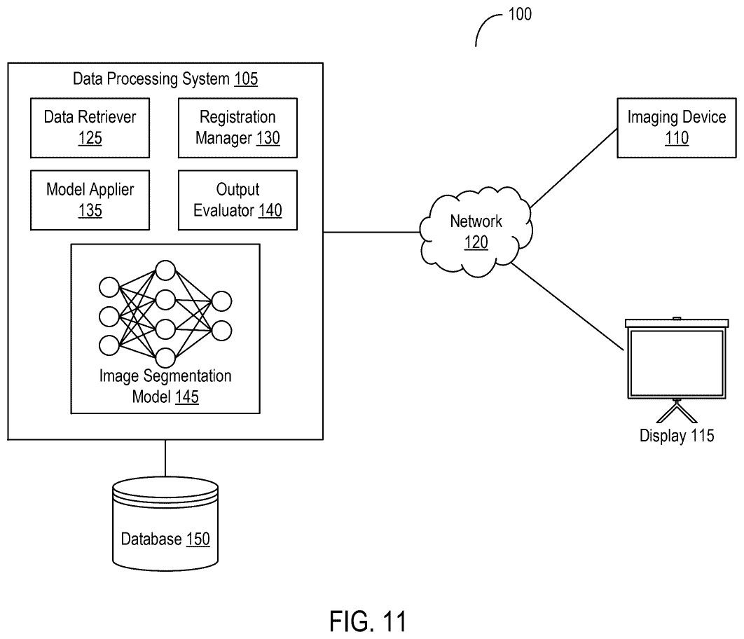

Referring now to FIG. 11, depicted is a block diagram of an example system 100 to determine tumor responses in brains from administering radiotherapy. In an overview, the system 100 can include at least one data processing system 105, at least one imaging device 110, at least one display 115, and at least one database 150 communicatively coupled with another via at least one network 120. The data processing system 105 can include at least one data retriever 125, at least one registration manager 130, at least one model applier 135, at least one output evaluator 140, and at least one image segmentation model 145 (generally referred to as the ISM or “machine learning (ML) model” herein), among others. Each of the components in the system 100 (e.g., the data processing system 105 and the display 115) as detailed herein may be implemented using hardware (e.g., one or more processors coupled with memory), or a combination of hardware and software as detailed herein in Section C.

In further detail, the data processing system 105 may (sometimes herein generally referred to as a computing system or a server) be any computing device including one or more processors coupled with memory and software and capable of performing the various processes and tasks described herein. The data processing system 105 can be in communication with the imaging device 110, display 115, the database 150, and other devices, via the network 120. The data processing system 105 may be situated, located, or otherwise associated with at least one server group. The server group may correspond to a data center, a branch office, or a site at which one or more servers corresponding to the data processing system 105 are situated. The data processing system 105 can perform or implement any of the functionalities detailed herein in conjunction with Section A (e.g., including the functionalities of METRO).

In the data processing system 105, the data retriever 125 can identify images. In some embodiments, the data retriever 125 can identify images including annotated information for a subject with a condition associated with the identified images. The registration manager 130 can perform an image registration on images identified by the data retriever 125. The model applier 135 can apply the ISM 145 to detect a segment identifying a region of interest within a biomedical image. The output evaluator 140 may determine a metric based on one or more biomedical images and transmit the metric to the display 115 (and/or store in the database 150). In some embodiments, the metric includes a plurality of data points obtained from a plurality of time points. For example, the metric may include a diameter, a volume, a dimension, etc., connecting between different time points (e.g., FIG. 10E).

The ISM 145 can be any type of machine learning (ML) algorithm or model to detect a segment identifying a region of interest (ROI) from one or more biomedical images. The ISM 145 can be maintained on the data processing system 105. The ISM 145 can be, for example, a deep learning artificial neural network (ANN), such as an encoder-decoder model with a convolution neural network architecture. In general, the ISM 145 can have one or more biomedical images in any modality from a subject as an input, an identification of the ROI within the one or more biomedical images as an output, and a set of weights relating the input to the output, among others. The ISM 145 may have been initialized, trained, and established using a training dataset in accordance with learning techniques (e.g., supervised or semi-supervised). The training dataset can include or identify a set of examples. Each example can include a respective biomedical image and an annotation defining the ROI within the respective biomedical image.

The imaging device 110 (sometimes herein generally referred to as an imaging scanner or an image acquirer) may be any device to acquire biomedical images of subjects. The biomedical image can be acquired in accordance with an imaging technique, such as a magnetic resonance imaging (MRI) scanner, a nuclear magnetic resonance (NMR) scanner, high-energy electromagnetic radiation (X-ray), computed tomography (CT) scanner, an ultrasound imaging scanner, a positron emission tomography (PET) scanner, or a photoacoustic spectroscopy scanner, among others. Although primarily discussed herein in terms of an MRI, other imaging modalities besides those listed above may be supported by the data processing system 105. The imaging device 110 can be in communication with the data processing system 105 and the display 115 to provide acquired projection images.

The display 115 (sometimes herein referred to as an operator device or a clinician device) can be any computing device comprising one or more processors coupled with memory and software and capable of providing an output projection image. The display 115 can be associated with an entity (e.g., a clinician) examining the subject or biomedical images from the subject. The display 115 can be in communication with the data processing system 105 and the imaging device 110 to exchange data. The display 115 can display projection images acquired from the imaging device 110. The display 115 can be used to input to create text reports for the projection images.

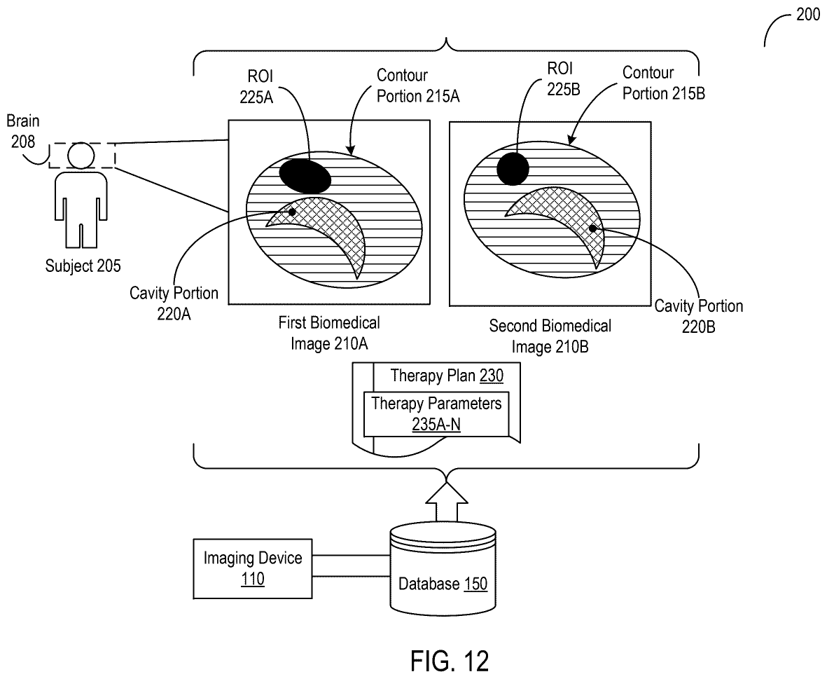

Referring now to FIG. 12, depicted is a block diagram of an example process 200 for identifying, for a subject 205 diagnosed with cancer, a plurality of biomedical images of a brain 208 of the subject 205. The process 200 can include or correspond to operations performed in the system 100 to identify the plurality of biomedical images (e.g., a first biomedical image 210A, a second biomedical image 210B, etc.). The subject 205 may have, may be at risk of, or may be afflicted with at least one cancer affecting at least a portion of a brain 208 of the subject 205. The subject 205 may be a human or animal subject, among others. In some embodiments, the cancer may include a metastasized cancer that has spread to the brain. For example, the metastasized cancer may include any primary site, such as at least one of colorectal cancer, lung cancer, breast cancer, ovarian cancer, prostate cancer, uterine cancer, or thyroid cancer. The metastasized cancer may have originated from another organ and spread to the brain 208 in the subject 205. In some embodiments, the cancer may include a primary cancer.

To address, alleviate, or otherwise treat the cancer in the brain 208 in the subject 205, the subject 205 may be administered with a radiotherapy over one or more time instances. The radiotherapy can include at least one of stereotactic radiosurgery (SRS) (e.g., frameless single-fraction or hypofractionated stereotactic radiosurgery), a brachytherapy, or a proton radiotherapy, among others. The radiotherapy may be administered to the subject 205 via a radiotherapy administration device, such as a linear accelerator (LINAC), a radiosurgery device, a proton therapy device, or a brachytherapy device, among others.

The data retriever 125 executing on the data processing system 105 can retrieve, identify, or otherwise obtain a set of biomedical images 210A and 210B (generally referred to herein as biomedical images 210) of the brain 208 of the subject 205. The biomedical images 210 may be retrieved from the database 150 or directly from the imaging device 110. Each biomedical image 210 may be acquired by the imaging device 110 in accordance with the imaging technique (e.g., MRI). The imaging device 110 can acquire the set of biomedical images 210 of the brain 208 of the subject 205 over a plurality of time instances in at least partial concurrence with the administration of the subject 205 with one or more radiotherapy instances. Upon acquisition, the imaging device 110 may store and maintain the biomedical images 210 on the database 150 to be accessed by the data retriever 125. The biomedical images 210 may be stored and maintained as image files on the database 150, such as Digital Imaging and Communications in Medicine (DICOM) files or Neuroimaging Informatics Technology Initiative (NIfTI) files, among others.

Each biomedical image 210 may be acquired at a respective time instance. The first biomedical image 210A may be acquired at a first time instance. The second biomedical image 210B may be acquired at a second time instance. The second time instance may be subsequent to the first time instance, after a predetermined time period. In some embodiments, the second time instance may be subsequent to at least one administration of radiotherapy to the brain 208. In some embodiments, the first time instance may be prior to administration of the radiotherapy. In some embodiments, the first time instance may also be subsequent to another administration of radiotherapy to the brain 208, prior to the administration associated with the second time instance. The first biomedical image 210A may include at least one contour portion 215A, at least one cavity portion 220A, and at least one region of interest (ROI) 225A at the first time instance, among others. Likewise, the second biomedical image 210B may include at least one contour portion 215B, at least one cavity portion 220B, and at least one ROI 225B at the second time instance. The contour portions 215A and 215B may correspond to a structure in the brain 208 at the first and second time instances respectively. For example, the contour portions 215A and 215B may correspond to a contour of the brain 208, such as anterior edge of longitudinal fissure, an anterior edge of the brain stem, an edge of primary cerebellar fissure, an edge of the longitudinal fissure, a bottom edge of a left or right temporal lobe, an edge (e.g., a superior or posterior) edge of a left or right lateral ventricle, among others. The cavity portions 220A and 220B may correspond to another structure in the brain 208 at the first and second time instances respectively. For instance, the cavity portion 220A and 220B may correspond to a superior edge of lateral ventricles, an inferior edge of lateral ventricles, or posterior edge of brainstem, among others. The ROI 225A and 225B may correspond to a tumor (or another feature of interest) within the brain 208 at the first and second time instances respectively.

In addition, the data retriever 125 may retrieve, identify, or otherwise obtain at least one therapy plan 230 associated with the administration of the radiotherapy to the subject 205. The therapy plan 230 may identify, specify, or otherwise define one or more therapy parameters 235A-N (hereinafter generally referred to as therapy parameters 235). The one or more therapy parameters 235 of the therapy plan 230 may define the administration of the radiotherapy to the tumor (e.g., corresponding to the ROI 225A or 225B) in the brain 208 of the subject 205. For example, the therapy parameters 235 may define or identify the subject 205, the type of radiotherapy, type of cancer affecting the brain 208, a dosage corresponding to amount of radiation applied to the tumor, and a location of the tumor (e.g., coordinates within each biomedical image 210), among others. The therapy plan 230 may be stored and maintained on the database 150 as one or more files, such as extensible markup file (XML), a log file, a comma-separated value (CSV) file, or a JavaScript Object Notation (JSON) file, among others.

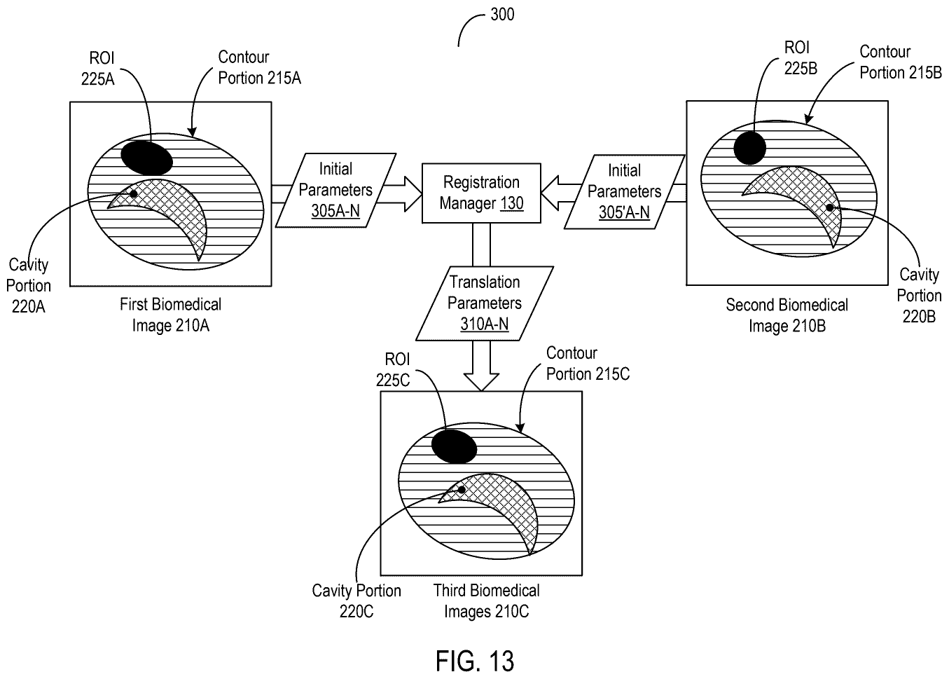

Referring now to FIG. 13, depicted is a block diagram of an example process 300 for performing an image registration. The process 300 can include or correspond to operations performed in the system 100 to perform an image registration. Under the process 300, the registration manager 130 executing on the data processing system 105 can perform an image registration on the first biomedical image 210A with the second biomedical image 210B. Although described and depicted with respect to two biomedical images, any number of biomedical images may be processed or used. The image registration may be based on correspondences in brain structures as depicted in the first biomedical images 210A and second biomedical image 210B. The image registration may be based on a correspondence between the contour portion 215A in the first biomedical image 210A and the contour portion 215B in the second biomedical image 210B for the associated structure (e.g., brain parenchyma). The image registration may be based on a correspondence between the cavity portion 220A in the first biomedical image 210A and the cavity portion 220B in the second biomedical image 210B for the associated structure (e.g., lateral ventricle). In some embodiments, the registration manager 130 can align two or more biomedical images 210 of the brain 208 taken at different times and from different viewpoints, among others, thereby aligning the resultant images so that corresponding structures in the images coincide spatially.

Based on the correspondences between the structures, the registration manager 130 can calculate, generate, or otherwise determine a set of translation parameters 310A-N (hereinafter generally referred to as translation parameters 310). The translation parameters 310 may specify, identify, or otherwise define transformations to align the contour portion 215A and the cavity portion 220A of the first biomedical image 210A with the contour portion 215B and the cavity portion 220B of the second biomedical image 210B, or vice-versa. The registration manager 130 can determine the translation parameters 310 based on the correspondence between the contour portion 215A) and the contour portion 215B for the corresponding structure (e.g., the contour of the brain 208). The registration manager 130 can determine the translation parameters 310 based on the correspondence between the cavity portion 220A and the cavity portion 220B for the corresponding structure (e.g., the lateral ventricle). In determining the correspondences, the registration manager 130 can define, identify, or otherwise use one of the biomedical images 210 as the reference image. In some embodiments, the registration manager 130 can select or identify the biomedical image 210 acquired earlier as the reference image (e.g., the first biomedical image 210A). Although the first biomedical image 210A is primarily discussed herein as the reference image, in some embodiments, the registration manager 130 can select or identify the biomedical image 210 acquired at a later stage as the reference image (e.g., the second biomedical image 210B).

In some embodiments, to facilitate the image registration, the registration manager 130 can detect the contour portion 215A and the cavity portion 220A from the first biomedical image 210A and the contour portion 215B and the cavity portion 220B from the second biomedical image 210B. In some embodiments, the registration manager 130 may use one or more image segmentation models (e.g., the ISM 145) to detect the portions within the respective biomedical images 210. The image segmentation models may have trained using a set of examples in accordance with supervised, unsupervised, or weakly supervised learning. Each example may include a biomedical image (e.g., in a similar or same MRI modality) of a brain of a respective subject and an annotation defining locations (e.g., pixel coordinates) of the structure (e.g., contour and cavity structures) in the biomedical image. By applying each biomedical image 210 to the trained image segmentation models, the registration manager 130 can detect the cavity portion 220 and the contour portion 220 from the respective biomedical image 210. In some embodiments, each biomedical image 210 may include or identify an annotation defining locations (e.g., pixel coordinates) of the contour portion 215 and the cavity portion 220. The annotation may have been generated or entered by a clinician examining the biomedical image 210. From the annotation, the registration manager 130 can detect the cavity portion 220 and the contour portion 220 from the biomedical image 210. Using the detected portions, the registration manager 130 can identify or determine the correspondences between the structures across the biomedical images 210A and 210B.

In performing the image registration, the registration manager 130 can calculate, determine, or otherwise generate a set of initial translation parameters (e.g., initial parameters 305A-N for the first biomedical image 210A, initial parameters 305′A-N for the second biomedical image 210B (hereinafter generally referred to as initial parameters 305 and 305′). In some embodiments, the registration manager 130 can calculate the initial parameters 305 for the first biomedical image 210A with the second biomedical image 210B as a reference. In some embodiments, the registration manager 130 can calculate the initial parameters 305′ for the second biomedical image 210B with the first biomedical image 210A as a reference. Although depicted as receiving both the initial parameters 305 and 305′ it should be understood that the registration manager 130 can perform registration based on one of the initial parameters 305 and 305′. To generate the set of initial translation parameters 305, the registration manager 130 can identify, calculate, or otherwise determine moments for the first biomedical image 210A. In addition, the registration manager 130 can identify, calculate, or otherwise or determine moments for the first biomedical image 210B to generate the set of initial translation parameters 305′. The moments may characterize or define distribution of pixel intensities within the respective biomedical image 210. The moments may include, for example, area or total intensity moment, or a centroid moment, a covariance moment, among others. The registration manager 130 can use the set of moments for the first biomedical image 210A as the initial set of translation parameters 305. The registration manager 130 can use the set of moments for the second biomedical image 210B as the initial set of translation parameters 305′.

In some embodiments, the registration manager 130 can generate, calculate, or otherwise determine the second set of initial translation parameters 305′ to align a portion (e.g., the contour portion 215A or cavity portion 220A) in the first biomedical image 210A with a corresponding portion (e.g., the contour portion 215B or cavity portion 220B) in the second biomedical image 210B. The alignment may include transformations of the second biomedical images 210B to match or correspond the structures depicted in the second biomedical images 210B with the structures in the first biomedical image 210A. The transformations for the alignment may include, for example, rotation, scaling, moving, or affine transformations, among others. In some embodiments, the registration manager 130 can modify the second set of initial translation parameters 305′ to align structures with the first set of initial of parameters 305. To determine the second set of initial translation parameters 305′, the registration manager 130 can identify or determine transformations to align the contour portion 215A of the first biomedical image 210A with the contour portion 215B of the second biomedical image 210B. Here, cach of the contour portions 215 can correspond to a respective contour of a parenchyma of the brain 208 in the subject 205. With the determination, the registration manager 130 can adjust, change, or otherwise modify the set of initial translation parameters 305′ to generate the set of translation parameters 310 to align or correspond the cavity portion 220A of the first biomedical image 210A with the cavity portion 220B of the second biomedical image 210B. Here, each of the cavity portions 220 can correspond to a lateral ventricle of the brain 208 in the subject 205.

In accordance with the set of translation parameters 310, the registration manager 130 can create, produce, or otherwise generate at least one third biomedical image 210C using the second biomedical image 210B. The registration manager 130 may apply the transformations defined by the set of translation parameters 310 on the second biomedical image 210B to produce the third biomedical image 210C. The third biomedical image 210C may include at least one contour portion 215C, at least one cavity portion 220C, and at least one ROI 225C at the second time instance. The contour portion 215C may correspond to the contour portion 215B, modified in accordance with the set of translation parameters 310. The cavity portion 220C may correspond to the cavity portion 220B, modified in accordance with the set of translation parameters 310. The ROI 225C may correspond to the ROI 225B and by extension the tumor in the brain 208 of the subject 205 at the second time instance. Although the image registration did not target the ROI 225, the ROI 225C may be modified as a result of the transformations specified in the set of translation parameters 310. Although the third biomedical image 210C is described primarily herein as generated using the second biomedical image 210B using the set of translation parameters 310, in some embodiments, the registration manager 130 may generate the third biomedical image 210C from the first biomedical image 210B, a combination of the first biomedical image 210A and the second biomedical image 210B, or any plural number of biomedical images.

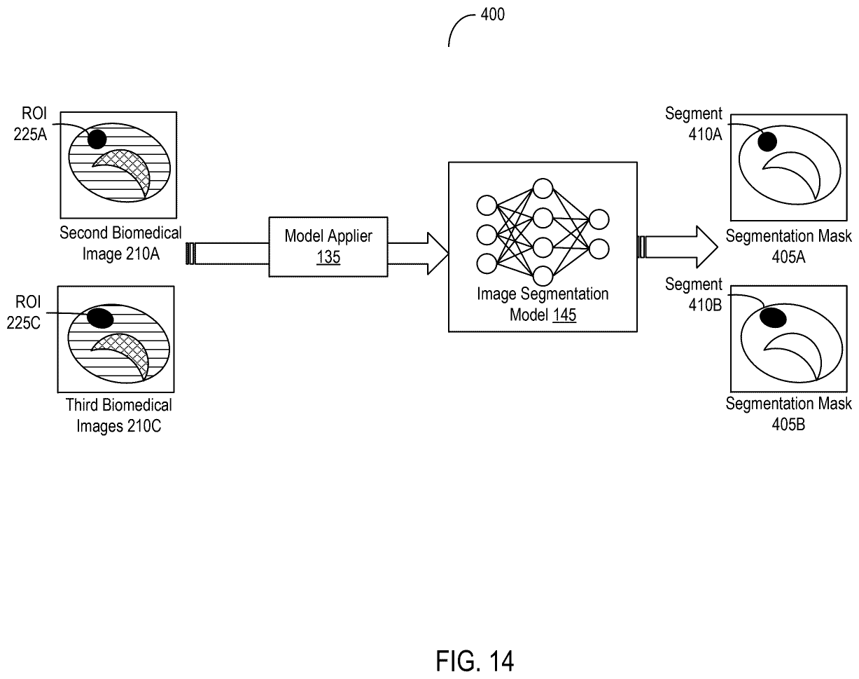

Referring now to FIG. 14, depicted is a block diagram of an example process 400 for detecting a segment identifying an ROI. The process 400 can include or correspond to operations performed in the system 100 to perform a detection of the segment using the ISM 145. Although described and depicted with respect to two biomedical images, any number of biomedical images may be processed or used. Under the process 400, the model applier 135 may use the ISM 145 to detect segments corresponding to the ROI 225 in each respective biomedical image 210. The model applier 135 may feed or apply the first biomedical image 210A and the biomedical image 210C to the ISM 145. By applying, the model applier 135 may process cach biomedical image 210 in accordance with the set of weights of the ISM 145. From processing the first biomedical image 210A using the set of weights of the ISM 145, the model applier 135 may output, produce, or otherwise generate at least one segmentation mask 405A. Likewise, from processing the third biomedical image 210C using the set of weights of the ISM 145, the model applier 135 may output, produce, or otherwise generate at least one segmentation mask 405C. The segmentation mask 405A may define or identify at least one segment 410A (also referred herein as a gross tumor volume (GTV)) identifying the ROI 225A in the first biomedical image 210A. The segmentation mask 405C may define or identify at least one segment 410B (also referred herein as a gross tumor volume (GTV)) identifying the ROI 225C in the third biomedical image 210C.

In some embodiments, the ISM 145 may have been initialized, trained, and established using a training dataset. The training dataset may include or identify a set of examples. Each example may include (i) a respective sample biomedical image of a brain of a respective subject and having at least one ROI corresponding to a tumor in the brain. Each example may include a respective annotation identifying a corresponding segment identifying the respective ROI. For instance, each example may be acquired or derived from a different subject. The ISM 145 may be trained in accordance with supervised, unsupervised, or weakly supervised training methods, among others. For instance, under the supervised training method, the biomedical image from each example may be applied to the ISM 145 to generate a segmentation mask defining the ROI (e.g., using pixel locations). The ROI defined by the segmentation mask may be compared against the ROI as defined by the annotation. Based on the comparison, a loss metric (e.g., as mean squared error (MSE), mean absolute error (MAE), cross-entry loss, Hinge loss, Huber loss, or L-norm loss) may be calculated to indicate a degree of deviation of the ROI outputted by the ISM 145 relative to the expected ROI as defined by the annotation. The loss metric may be used to update the weights of the ISM 145.

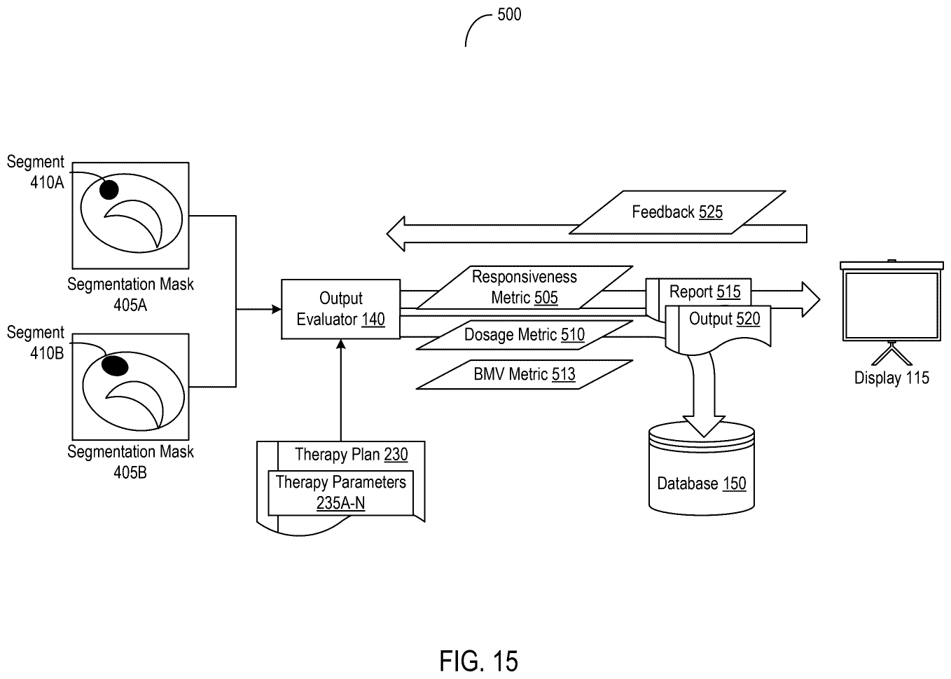

Referring now to FIG. 15, depicted is a block diagram of an example process 500 for determining a responsiveness metric. The process 500 can include or correspond to operations performed in the system 100 to determine a responsiveness metric 505 or a dosage metric 510. Under the process 500, the output evaluator 140 executing the data processing system 105 can calculate, generate, or otherwise determine at least one of the responsiveness metric 505 for the subject 205 based on the first segment 410A and the second segment 410B. The responsiveness metric 505 may identify, define, or otherwise indicate a degree of responsiveness of the tumor in the subject 205 to the administration of the radiotherapy to the brain 208 of the subject 205. For example, the responsiveness metric 505 can measure, rate, or identify how well the tumor in the subject 205 is responsive to the administration of the radiotherapy to the brain 208.

In some embodiments, the output evaluator 140 can determine the responsiveness metric 505 based on a difference in size (e.g., lateral, longitudinal, diagonal, or longest segment) between the first segment 410A and the second segment 410B. The difference in size may correlate or correspond to an effectiveness of the administration of the radiotherapy to the brain 208 across the time instances. For example, the output evaluator 140 can compare a longitudinal size of the first segment 410A and a longitudinal size of the second segment 410B to calculate the difference in longitudinal size between the first segment 410A and the second segment 410B. In some embodiments, the output evaluator 140 can determine the responsiveness metric 505 based on a difference in a dimension, a shape, an orientation, a size, between the first segment 410A and the second segment 410B.

In some embodiments, the output evaluator 140 can calculate, generate, or otherwise determine at least one dosage metric 510 for the subject 205 based on the set of therapy parameters 235 of the therapy plan 230. The dosage metric 510 may identify, define, or otherwise indicate a dosage of the radiotherapy received by the tumor in the brain 208 in the subject 205. From the therapy parameters 235, the output evaluator 140 may identify the tumor targeted for the administration of the radiotherapy in the brain 208 of the subject 205. The output evaluator 140 can identify or determine whether the tumor targeted by the therapy parameters 235 of the therapy plan 230 corresponds to the tumor identified by the segment 410A or 410B. To determine, the output evaluator 140 can compare the location of the segment 410A or 410B with the location of the tumor defined by the therapy parameters 235 of the therapy plan 230.

When the location of the segment 410A or 410B corresponds to (e.g., greater than or equal to 90% overlap) the location of the tumor defined by the therapy parameters 235, the output evaluator 140 can identify or determine that the tumor identified by the segment 410A or 410B as targeted by the administration of the radiotherapy. The output evaluator 140 can determine the dosage metric 510 based on the dosage identified in the dosage of the therapy parameters 235 of the therapy plan 230. In some embodiments, the output evaluator 140 can determine the dosage metric 510 as a function of at least one of the segment 410A or 410B or the dosage of the therapy parameters 235. For example, the output evaluator 140 can determine the dosage metric 510 based on the defined dosage, adjusted by the change in size of the segments 410A and 410B.

Conversely, when the location of the segment 410A or 410B does not correspond to (e.g., less than 90% overlap) the location of the tumor defined by the therapy parameters 235, the output evaluator 140 can identify or determine that the tumor identified by the segment 410A or 410B as not targeted by the administration of the radiotherapy. The output evaluator 140 can determine the dosage metric 510 as a function of at least one of the segment 410A or the 410B. For instance, the output evaluator 140 can determine the dosage metric 510 based on the change in size of the segments 410A and 410B. In some embodiments, the output evaluator 140 can determine the dosage metric 510 as a function of the dosage of the therapy parameters 235 and a distance of the targeted tumor from the segment 410A or 410B. For example, the output evaluator 140 can determine the dosage metric 510 based on the defined dosage, adjusted by the distance (e.g., centroid distance) between the targeted tumor and the tumor associated with the segment 410A or 410B.

With the generation of the responsiveness metric 505, the output evaluator 140 may store and maintain an association between the subject 205 (e.g., using an anonymous identifier) and the responsiveness metric 505 on the database 150, using one or more data structures. The output evaluator 140 may store and maintain the association of the subject 205 with one or more of the responsive metric 505, the dosage metric 510, the biomedical images 210, the therapy plan 230, the segmentation masks 405, or the segments 410, among others. The data structures may include, for example, an array, a linked list, a stack, a tree, a hash table, among others.

The output evaluator 140 can create, produce, or otherwise generate at least one report 515 identifying the tumor and the responsiveness metric 505. In some embodiments, the output evaluator 140 can include the dosage metric 510 in the report 515. The report 515 may also include or identify, for example, with one or more of the responsive metric 505, the dosage metric 510, the biomedical images 210, the therapy plan 230, the segmentation masks 405, or the segments 410, among others. The report 515 may include or identify information regarding the administration of the radiotherapy to the brain 208 of the subject 205. The output evaluator 140 can send, transmit, or otherwise provide, for presentation on the display 115, the report 515 identifying the tumor and the metrics (e.g., the responsiveness metric 505 and the dosage metric 510). Upon receipt, the display 115 may present the information from the report 515 to a clinician examining the subject 205. The subsequent administration of the radiotherapy may be adjusted or modified to enhance efficacy of the treatment. The additional radiotherapy may be administered to the tumor in the subject 205, as identified by at least one of the segment 410A or 410B of the segmentation mask 405A or 405B respectively. For instance, the clinician may orient or move the beam emitter used for radiotherapy based on the segment 410B of the segmentation mask 405B, and then provide the radiotherapy via the beam emitter to the tumor of the subject 205. Further administration of the radiotherapy to the subject 205 may be halted, upon determination that the radiotherapy has been successful.

In some embodiments, the output evaluator 140 can calculate, generate, or otherwise determine at least one brain metastasis velocity (BMV) metric 513 of the tumor (e.g., metastasized tumor) within the brain 208 of the subject 205. The BMV metric 513 may include a cumulative number of new brain metastases that have developed since the first administration of radiotherapy (e.g., stereotactic radiosurgery (SRS)). For example, the BMV metric 513 may be or include a parameter defined as:

$${BMV}_{Time ( i )} = \frac{Cumulative number of new brain metastases since initial SRS}{Total time ( y ) between initial SRS and Time ( i )} .$$

In some examples, the output evaluator 140 can determine the BMV metric 513 of the disease across the first time instance and the second time instance, based on the first segment 410A and the second segment 410B. The BMV metric 513 may indicate a rate of change in tumor burden over time and may be derived based on the first segment 410A and the second segment 410B, which respectively identify the tumor at the first and second time instances. For example, the BMV metric 513 may be calculated as a function of the number of new lesions detected between the time instances, divided by the elapsed time between acquisitions of the first biomedical image 210A and the second biomedical image 210B. In some implementations, the BMV metric 513 may alternatively or additionally account for a volume growth rate or a lesion expansion velocity. In some examples, the BMV metric 513 may be generated in parallel with the responsiveness metric 505 and/or the dosage metric 510.

The output evaluator 140 may include the BMV metric 513 in the report 515 or generate an output 520 for transmission to the display 115 and/or the database 150. The report 515 or the output 520 may include an association between the BMV metric 513 and the corresponding tumor, subject 205, and other metrics such as the responsiveness metric 505. In some examples, the BMV metric 513 may be used by a clinician to assess aggressiveness of disease, potential prognosis, or the need for additional monitoring. In some embodiments, the output evaluator 140 may classify patients into a plurality of groups based on the BMV metric 513. For example, the patients can be separated into a first group having a lower number of the BMV metric 513 (e.g., <4 new metastases/year), a second group having an intermediate number of the BMV metric 513 (e.g., 4-13 new metastases/year), and a third group having a higher number of the BMV metric 513 (e.g., >13 new metastases/year). The classifications may be provided for presentation and may be used to evaluate candidate treatments.

In some embodiments, the output evaluator 140 may retrieve, obtain, or otherwise receive feedback 525 from the display 115 (or the computing device associated with the display 115). Upon receipt of the report 515, the display 115 may present, render, or otherwise display the information, including the segmentation masks 405. The user (e.g., the clinician examining the subject) may interact with the presentation of the segmentation tasks 115 on the display 115 (e.g., using an input/output device associated with the computing device). For instance, the user may specify that the segmentation masks 405 are to be expanded or contracted, or may confirm the accuracy of the segmentation. The computing device associated with the display 115 may produce, create, or otherwise generate feedback 525 based on the user interaction. The feedback 525 may specify, define, or otherwise identify one of a modification (e.g., expansion or contraction) of the segmentation masks 405 or a confirmation (e.g., indication of accuracy) of the segmentation masks 405. With the generation of the feedback 525, the computing device may transmit, send, or otherwise provide the feedback 525 to the output evaluator 140. The output evaluator 140 in turn may receive the feedback 525.

Using the feedback 525, the output evaluator 140 may perform any number of actions. In some embodiments, the output evaluator 140 may update the ISM 145 based on the feedback 525. For example, the output evaluator 140 may determine a loss metric (e.g., as mean squared error (MSE), mean absolute error (MAE), cross-entry loss, Hinge loss, Huber loss, or L-norm loss) based on the modifications of the segmentation masks 405 identified in the feedback 525 and the segmentation masks 405 generated by the ISM 145. In accordance with the loss metric, the output evaluator 140 may update the weights of the ISM 145. In some embodiments, the output evaluator 140 may redetermine the responsiveness metric 505, the dosage metric 510, or the BMV metric 513 using the feedback 525. The output evaluator 140 may redetermine the responsiveness metric 505, the dosage metric 510, or the BMV metric 513 using the modified segmentation masks 405 identified in the feedback 525, in a similar manner as described herein. The output evaluator 140 may provide the updated responsiveness metric 505, dosage metric 510, or BMV metric 513 for presentation via the display 115.

By performing the image registration and using the ISM 145, the data processing system 105 may generate and provide more accurate and meaningful metrics indicating responsiveness of tumors in the brain 208 to one or more administrations of radiotherapy. For example, the subsequent administration of radiotherapy may be determined and adjusted (e.g., based on the report 515) to be more accurate (and thus more effective) at treating and regressing the tumors in the brain 208. By providing the more accurate and meaningful metrics based on the processes discussed herein, the subsequent administration of radiotherapy may be improved. For example, the radiotherapy may be targeted to a more accurate location, thereby enabling more effective treatment. The information may be used to decide whether to administer the subject with additional radiotherapy at a subsequent time instances. Furthermore, with the increased efficacy of treatment, the overall cost to treat the tumors in the brain 208, such as radiotherapy, computing resources that would have otherwise been consumed in relatively fewer effective treatments, may be conserved. For example, if the prior administrations of the radiotherapy treatment are determined to be successful, additional radiotherapy treatment may be halted, thereby saving resources spent for the radiotherapy and consumption of computing resources (e.g., processor and memory).

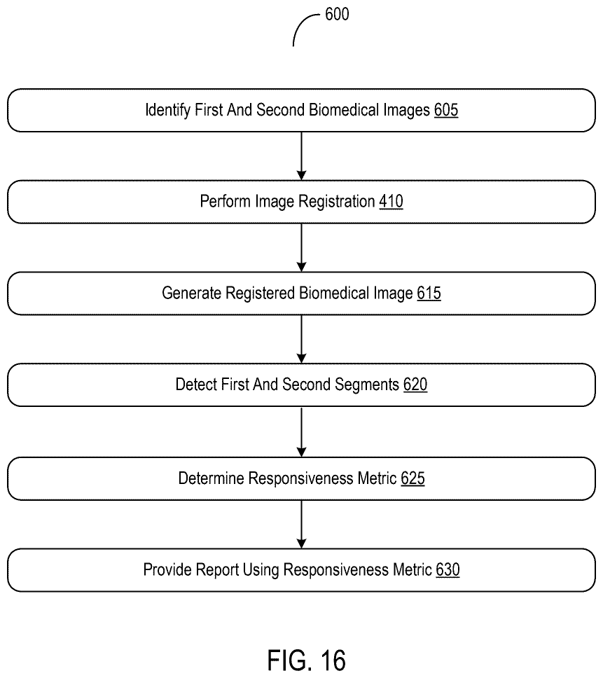

Referring now to FIG. 16, depicted is an illustrative flow diagram of an example method 600 for determining tumor responses in brains from administering radiotherapy. The method 600 can be executed, performed, or otherwise carried out by the system 100. Under the method 600, a first biomedical image (e.g., the first biomedical image 210A) and a second biomedical image (e.g., the second biomedical image 210B) may be identified (605). A computing system (e.g., the data processing system 105) may perform an image registration (610). The computing system may generate a registered biomedical image (615). The computing system may detect first and second segments (620). The computing system may determine a responsiveness metric (625). The computing system may provide a report using the responsiveness metric (630).

### C. Computing and Network Environment

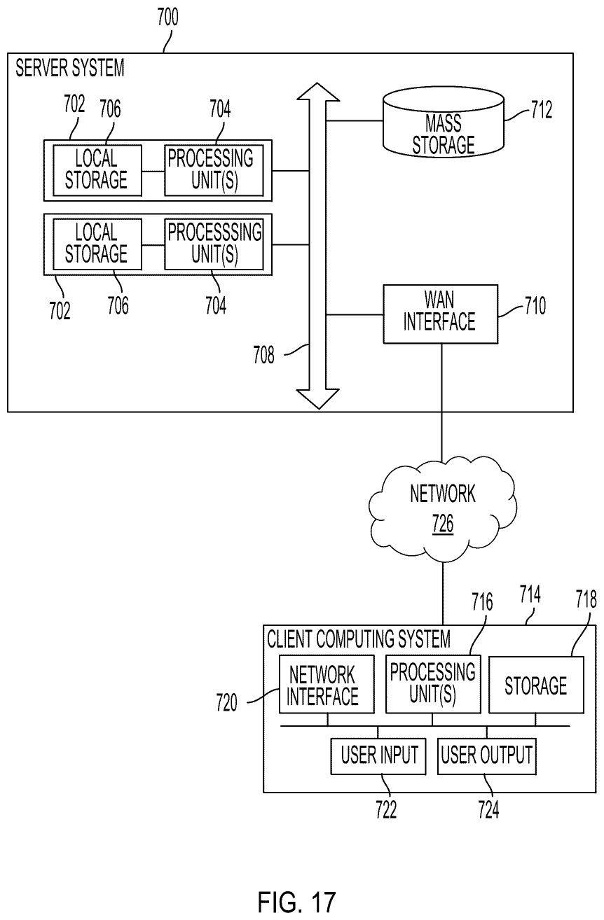

Various operations described herein can be implemented on computer systems. FIG. 17 shows a simplified block diagram of an example of a representative server system 700, client computing system 714, and network 726 usable to implement certain embodiments of the present disclosure. In various embodiments, server system 700 or similar systems can implement services or servers described herein or portions thereof. Client computing system 714 or similar systems can implement clients, described herein. The systems 100 described herein can be similar to the server system 700. Server system 700 can have a modular design that incorporates a number of modules 702 (e.g., blades in a blade server embodiment); while two modules 702 are shown, any number can be provided. Each module 702 can include processing unit(s) 704 and local storage 706.

Processing unit(s) 704 can include a single processor, which can have one or more cores, or multiple processors. In some embodiments, processing unit(s) 704 can include a general-purpose primary processor as well as one or more special-purpose co-processors, such as graphics processors, digital signal processors, or the like. In some embodiments, some or all processing units 704 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 704 can execute instructions stored in local storage 706. Any type of processors in any combination can be included in processing unit(s) 704.

Local storage 706 can include volatile storage media (e.g., DRAM, SRAM, SDRAM, or the like) and/or non-volatile storage media (e.g., magnetic or optical disk, flash memory, or the like). Storage media incorporated in local storage 706 can be fixed, removable, or upgradeable as desired. Local storage 706 can be physically or logically divided into various subunits, such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 704 need at runtime. The ROM can store static data and instructions that are needed by processing unit(s) 704. The permanent storage device can be a non-volatile read-and-write memory device that can store instructions and data even when module 702 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 706 can store one or more software programs to be executed by processing unit(s) 704, such as an operating system and/or programs implementing various server functions such as functions of the system 100 or any other system described herein, or any other server(s) associated with system 100 or any other system described herein.

“Software” refers generally to sequences of instructions that, when executed by processing unit(s) 704, cause server system 700 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 704. Software can be implemented as a single program or a collection of separate programs or program modules that interact as desired. From local storage 706 (or non-local storage described below), processing unit(s) 704 can retrieve program instructions to execute and data to process in order to execute various operations described above.

In some server systems 700, multiple modules 702 can be interconnected via a bus or other interconnect 708, forming a local area network that supports communication between modules 702 and other components of server system 700. Interconnect 708 can be implemented using various technologies including server racks, hubs, routers, etc.

A wide area network (WAN) interface 710 can provide data communication capability between the local area network (interconnect 708) and the network 726, such as the Internet. Technologies can be used, including wired (e.g., Ethernet, IEEE 802.3 standards) and/or wireless technologies (e.g., Wi-Fi, IEEE 802.11 standards).

In some embodiments, local storage 706 is intended to provide working memory for processing unit(s) 704, providing fast access to programs and/or data to be processed while reducing traffic on interconnect 708. Storage for larger quantities of data can be provided on the local area network by one or more mass storage subsystems 712 that can be connected to interconnect 708. Mass storage subsystem 712 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 712. In some embodiments, additional data storage resources may be accessible via WAN interface 710 (potentially with increased latency).

Server system 700 can operate in response to requests received via WAN interface 710. For example, one of the modules 702 can implement a supervisory function and assign discrete tasks to other modules 702 in response to received requests. Work allocation techniques can be used. As requests are processed, results can be returned to the requester via WAN interface 710. Such operation can generally be automated. Further, in some embodiments, WAN interface 710 can connect multiple server systems 700 to each other, providing scalable systems capable of managing high volumes of activity. Other techniques for managing server systems and server farms (collections of server systems that cooperate) can be used, including dynamic resource allocation and reallocation.

Server system 700 can interact with various user-owned or user-operated devices via a wide-area network such as the Internet. An example of a user-operated device is shown in FIG. 17 as client computing system 714. Client computing system 714 can be implemented, for example, as a consumer device such as a smartphone, other mobile phone, tablet computer, wearable computing device (e.g., smart watch, eyeglasses), desktop computer, laptop computer, and so on.

For example, client computing system 714 can communicate via WAN interface 710. Client computing system 714 can include computer components such as processing unit(s) 716, storage device 718, network interface 720, user input device 722, and user output device 737. Client computing system 714 can be a computing device implemented in a variety of form factors, such as a desktop computer, laptop computer, tablet computer, smartphone, other mobile computing device, wearable computing device, or the like.

Processing unit(s) 716 and storage device 718 can be similar to processing unit(s) 704 and local storage 706 described above. Suitable devices can be selected based on the demands to be placed on client computing system 714; for example, client computing system 714 can be implemented as a “thin” client with limited processing capability or as a high-powered computing device. Client computing system 714 can be provisioned with program code executable by processing unit(s) 716 to enable various interactions with server system 700.

Network interface 720 can provide a connection to the network 726, such as a wide area network (e.g., the Internet) to which WAN interface 710 of server system 700 is also connected. In various embodiments, network interface 720 can include a wired interface (e.g., Ethernet) and/or a wireless interface implementing various RF data communication standards, such as Wi-Fi, Bluetooth, or cellular data network standards (e.g., 3G, 4G, LTE, etc.).

User input device 722 can include any device (or devices) via which a user can provide signals to client computing system 714; client computing system 714 can interpret the signals as indicative of particular user requests or information. In various embodiments, user input device 722 can include any or all of a keyboard, touch pad, touch screen, mouse or other pointing device, scroll wheel, click wheel, dial, button, switch, keypad, microphone, and so on.

User output device 737 can include any device via which client computing system 714 can provide information to a user. For example, user output device 737 can include display-to-display images generated by or delivered to client computing system 714. The display can incorporate various image generation technologies, e.g., a liquid crystal display (LCD), light-emitting diode (LED), including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that functions as both input and output device. In some embodiments, other user output devices 737 can be provided in addition to or instead of a display. Examples include indicator lights, speakers, tactile “display” devices, printers, and so on.

Some embodiments include electronic components, such as microprocessors, storage, and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operations indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 704 and 716 can provide various functionality for server system 700 and client computing system 714, including any of the functionality described herein as being performed by a server or client, or other functionality.

It will be appreciated that server system 700 and client computing system 714 are illustrative and that variations and modifications are possible. Computer systems used in connection with embodiments of the present disclosure can have other capabilities not specifically described here. Further, while server system 700 and client computing system 714 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. Embodiments of the disclosure can be realized using a variety of computer systems and communication technologies, including, but not limited to, specific examples described herein. Embodiments of the present disclosure can be realized using any combination of dedicated components, programmable processors, and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may refer to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media includes magnetic disk or tape, optical storage media such as compact disk (CD) or digital versatile disk (DVD), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

What is claimed is:

1. A method of determining tumor responses in brains from administering radiotherapy, comprising:
identifying, by one or more processors, for a subject diagnosed with cancer, a plurality of biomedical images of a brain of the subject, the plurality of biomedical images including:
(i) a first biomedical image acquired at a first time instance, the first biomedical image having (a) a first portion corresponding to a first structure of the brain, (b) a second portion corresponding to a second structure of the brain, and (c) a first region of interest (ROI) corresponding to a tumor within the brain at the first time instance;
(ii) a second biomedical image of the brain acquired at a second time instance subsequent to an administration of radiotherapy to the brain, the second biomedical image having (a) a third portion corresponding to the first structure, (b) a fourth portion corresponding to the second structure, and (b) a second ROI corresponding to the tumor within the brain at the second time instance;
performing, by the one or more processors, an image registration on the first biomedical image with the second biomedical image to determine a plurality of translation parameters, based on (i) a first correspondence between the first portion and the third portion for the first structure and (ii) a second correspondence between the second portion and the fourth portion for the second structure;
generating, by the one or more processors, a third biomedical image using the second biomedical image in accordance with the plurality of translation parameters;
detecting, by the one or more processors, using an image segmentation model, (i) a first segment identifying the first ROI within the third biomedical image and (ii) a second segment identifying the second ROI within the first biomedical image;
determining, by the one or more processors, a metric indicating a degree of responsiveness of the tumor in the subject to the administration of the radiotherapy to the brain, based on the first segment and the second segment; and
storing, by the one or more processors, using one or more data structures, an association between the subject and the metric.

2. The method of claim 1, further comprising:
identifying, by the one or more processors, the tumor as targeted for the administration of radiotherapy from a plurality of treatment parameters defining the administration of the radiotherapy to the tumor prior to the second time instance; and
determining, by the one or more processors, a second metric indicating a dose of the radiotherapy on the tumor based on at least one of the plurality of treatment parameters, the first segment and or the second segment, responsive to identifying the tumor as targeted for the administration of radiotherapy,
wherein storing the association further comprises storing the association among the subject, the tumor, the metric indicating the degree of responsiveness, and the second metric indicating the dose of the radiotherapy.

3. The method of claim 1, further comprising
identifying, by the one or more processors, the tumor corresponding to at least one of the first segment or the second segment as not targeted for the administration of radiotherapy, using a plurality of treatment parameters defining the administration of the radiotherapy; and
determining, by the one or more processors, a second metric indicating a dose of the radiotherapy on the tumor based on at least one of the first segment or the second segment, responsive to identifying the tumor as not targeted for the administration of radiotherapy, and
wherein storing the association further comprises storing the association among the subject, the tumor, the metric indicating the degree of responsiveness, and the second metric indicating the dose of the radiotherapy.

4. The method of claim 1, further comprising determining, by the one or more processors, a second metric indicating a brain metastasis velocity (BMV) of the tumor within the brain of the subject across the first time instance and the second time instance, based on the first segment and the second segment,
wherein storing the association further comprises storing the association among the subject, the tumor, the metric indicating the degree of responsiveness, and the second metric indicating the BMV of the tumor.

5. The method of claim 1, further comprising:
generating, by the one or more processors, a report identifying the tumor and the metric indicating the degree of responsiveness of the tumor to the administration of the radiotherapy; and
providing, by the one or more processors, for presentation, the report identifying the tumor and the metric.

6. The method of claim 1, wherein performing the image registration further comprises:
generating a first plurality of translation parameters based on a moment of intensities for the second biomedical image;
determining a second plurality of translation parameters to align the third portion in the second biomedical image with the first portion in the first biomedical image, the first portion and the third portion each corresponding to a respective contour of a parenchyma of the brain in the subject;
modifying the second plurality of translation parameters to generate the plurality of translation parameters to correspond the fourth portion of the second biomedical image with the second portion of the first biomedical image, the third portion and the second portion each corresponding to a lateral ventricle of the brain in the subject.

7. The method of claim 1, wherein performing the image registration further comprises detecting, using one or more image segmentation models, (i) the first portion and the second portion from the first biomedical image and (ii) the third portion and the fourth portion from the second biomedical image.

8. The method of claim 1, wherein determining the metric further comprises determining the metric based on a difference in longitudinal size between the first segment and the second segment.

9. The method of claim 1, wherein the image segmentation model is established using a plurality of examples, each example of the plurality of examples including (i) a respective sample biomedical image of a corresponding brain of a respective subject having a respective ROI corresponding to a respective tumor in the corresponding brain and (ii) a respective annotation identifying a corresponding segment identifying the respective ROI.

10. The method of claim 1, further comprising administering the tumor of the subject with the radiotherapy at a third time instance subsequent to the second time instance, as identified by at least one of the first segment or the second segment, wherein the cancer associated with the tumor comprises a metastasized cancer,
wherein the radiotherapy further comprises at least one of a stereotactic radiosurgery (SRS), a brachytherapy, a proton radiotherapy, or a whole brain radiation therapy (WBRT).

11. A system for determining tumor responses in brains from administering radiotherapy, comprising:
one or more processors coupled with memory, configured to:
identify, for a subject diagnosed with cancer, a plurality of biomedical images of a brain of the subject, the plurality of biomedical images including:
(i) a first biomedical image acquired at a first time instance, the first biomedical image having (a) a first portion corresponding to a first structure of the brain, (b) a second portion corresponding to a second structure of the brain, and (c) a first region of interest (ROI) corresponding to a tumor within the brain at the first time instance;
(ii) a second biomedical image of the brain acquired at a second time instance subsequent to an administration of radiotherapy to the brain, the second biomedical image having (a) a third portion corresponding to the first structure, (b) a fourth portion corresponding to the second structure, and (b) a second ROI corresponding to the tumor within the brain at the second time instance;
perform an image registration on the first biomedical image with the second biomedical image to determine a plurality of translation parameters, based on (i) a first correspondence between the first portion and the third portion for the first structure and (ii) a second correspondence between the second portion and the fourth portion for the second structure;
generate a third biomedical image using the second biomedical image in accordance with the plurality of translation parameters;
detect, using an image segmentation model, (i) a first segment identifying the first ROI within the third biomedical image and (ii) a second segment identifying the second ROI within the first biomedical image;
determine a metric indicating a degree of responsiveness of the tumor in the subject to the administration of the radiotherapy to the brain, based on the first segment and the second segment; and
store, using one or more data structures, an association between the subject and the metric.

12. The system of claim 11, wherein the one or more processors are configured to:
identify the tumor as targeted for the administration of radiotherapy from a plurality of treatment parameters defining the administration of the radiotherapy to the tumor prior to the second time instance; and
determine a second metric indicating a dose of the radiotherapy on the tumor based on at least one of the plurality of treatment parameters, the first segment and or the second segment, responsive to identifying the tumor as targeted for the administration of radiotherapy,
wherein the one or more processors are configured to store the association among the subject, the tumor, the metric indicating the degree of responsiveness, and the second metric indicating the dose of the radiotherapy.

13. The system of claim 11, wherein the one or more processors are configured to:
identify the tumor corresponding to at least one of the first segment or the second segment as not targeted for the administration of radiotherapy, using a plurality of treatment parameters defining the administration of the radiotherapy; and
determine a second metric indicating a dose of the radiotherapy on the tumor based on at least one of the first segment or the second segment, responsive to identifying the tumor as not targeted for the administration of radiotherapy, and
store the association among the subject, the tumor, the metric indicating the degree of responsiveness, and the second metric indicating the dose of the radiotherapy.

14. The system of claim 11, wherein the one or more processors are configured to:
determine a second metric indicating a brain metastasis velocity (BMV) of the tumor within the brain of the subject across the first time instance and the second time instance, based on the first segment and the second segment; and
store the association among the subject, the tumor, the metric indicating the degree of responsiveness, and the second metric indicating the BMV of the tumor.

15. The system of claim 11, wherein the one or more processors are configured to:
generate a report identifying the tumor and the metric indicating the degree of responsiveness of the tumor to the administration of the radiotherapy; and
provide for presentation, the report identifying the tumor and the metric.

16. The system of claim 11, wherein the one or more processors are configured to:
generate a first plurality of translation parameters based on a moment of intensities for the second biomedical image;
determine a second plurality of translation parameters to align the third portion in the second biomedical image with the first portion in the first biomedical image, the first portion and the third portion each corresponding to a respective contour of a parenchyma of the brain in the subject;
modify the second plurality of translation parameters to generate the plurality of translation parameters to correspond the fourth portion of the second biomedical image with the second portion of the first biomedical image, the third portion and the second portion each corresponding to a lateral ventricle of the brain in the subject.

17. The system of claim 11, wherein the one or more processors are configured to:
detect, using one or more image segmentation models, (i) the first portion and the second portion from the first biomedical image and (ii) the third portion and the fourth portion from the second biomedical image.

18. The system of claim 11, wherein the one or more processors are configured to determine the metric based on a difference in longitudinal size between the first segment and the second segment.

19. The system of claim 11, wherein the image segmentation model is established using a plurality of examples, each example of the plurality of examples including (i) a respective sample biomedical image of a corresponding brain of a respective subject having a respective ROI corresponding to a respective tumor in the corresponding brain and (ii) a respective annotation identifying a corresponding segment identifying the respective ROI.

20. The system of claim 11, wherein the tumor of the subject is administered with the radiotherapy at a third time instance subsequent to the second time instance, as identified by at least one of the first segment or the second segment, wherein the cancer associated with the tumor comprises a metastasized cancer, and
wherein the radiotherapy further comprises at least one of a stereotactic radiosurgery (SRS), a brachytherapy, a proton radiotherapy, or a whole brain radiation therapy (WBRT).
